(19) United States
(12) Patent Application Publication
(10) Pub. No.: US 2026/0182673 A1
(43) Pub. Date: Jul. 2, 2026

(54) SMOKING SUBSTITUTE SYSTEM

(71) Applicant: Imperial Tobacco Limited, Bristol (GB)

(72) Inventors: Kate Ferrie, Bristol (GB); Ross Shenton, Bristol (GB); Chris Lord, Bristol (GB); Med Benyezzar, Bristol (GB)

(21) Appl. No.: 19/396,260

(22) Filed: Nov. 20, 2025

Related U.S. Application Data
(63) Continuation of application No. 17/481,864, filed on Sep. 22, 2021, now Pat. No. 12,501,942.
Application No. 17/481,864 is a continuation of application No. PCT/EP2020/056783, filed on Mar. 13, 2020.
Application No. PCT/EP2020/056783 is a continuation of application No. PCT/EP2020/056790, filed on Mar. 13, 2020.
Application No. PCT/EP2020/056790 is a continuation of application No. PCT/EP2020/056794, filed on Mar. 13, 2020.
Application No. PCT/EP2020/056794 is a continuation of application No. PCT/EP2020/056795, filed on Mar. 13, 2020.
Application No. PCT/EP2020/056795 is a continuation of application No. PCT/EP2020/056798, filed on Mar. 13, 2020.
Application No. PCT/EP2020/056798 is a continuation of application No. PCT/EP2020/056800, filed on Mar. 13, 2020.
Application No. PCT/EP2020/056800 is a continuation of application No. PCT/EP2020/056801, filed on Mar. 13, 2020.
Application No. PCT/EP2020/056801 is a continuation of application No. PCT/EP2020/056803, filed on Mar. 13, 2020.
Application No. PCT/EP2020/056803 is a continuation of application No. PCT/EP2020/056804, filed on Mar. 13, 2020.
Application No. PCT/EP2020/056804 is a continuation of application No. PCT/EP2020/056810, filed on Mar. 13, 2020.
Application No. PCT/EP2020/056810 is a continuation of application No. PCT/EP2020/056812, filed on Mar. 13, 2020.
Application No. PCT/EP2020/056812 is a continuation of application No. PCT/EP2020/056817, filed on Mar. 13, 2020.
Application No. PCT/EP2020/056817 is a continuation of application No. PCT/EP2020/056827, filed on Mar. 13, 2020.
Application No. PCT/EP2020/056827 is a continuation of application No. PCT/EP2020/056833, filed on Mar. 13, 2020.
Application No. PCT/EP2020/056833 is a continuation of application No. PCT/EP2020/056834, filed on Mar. 13, 2020.
Application No. PCT/EP2020/056834 is a continuation of application No. PCT/EP2020/056835, filed on Mar. 13, 2020.
Application No. PCT/EP2020/056835 is a continuation of application No. PCT/EP2020/056841, filed on Mar. 13, 2020.
Application No. PCT/EP2020/056841 is a continuation of application No. PCT/EP2020/056852, filed on Mar. 13, 2020.

(30) Foreign Application Priority Data
Mar. 22, 2019 (EP) 19020139.2
Mar. 22, 2019 (EP) 19020144.2
Mar. 22, 2019 (EP) 19020145.9
Mar. 22, 2019 (EP) 19020146.7
Mar. 22, 2019 (EP) 19020162.4
Mar. 22, 2019 (EP) 19020174.9
Mar. 22, 2019 (EP) 19020192.1
Mar. 22, 2019 (EP) 19020196.2
Mar. 22, 2019 (EP) 19020198.8
Mar. 22, 2019 (EP) 19020202.8
Mar. 22, 2019 (EP) 19020215.0
Mar. 22, 2019 (EP) 19020217.6
Mar. 22, 2019 (EP) 19020221.8
Mar. 22, 2019 (EP) 19020222.6
Mar. 22, 2019 (EP) 19020224.2
Mar. 22, 2019 (EP) 19020226.7
Mar. 22, 2019 (EP) 19020227.5
Mar. 22, 2019 (EP) 19020228.3

(51) Int. Cl. A24F 40/90; A24F 40/20; A24F 40/51; A24F 40/53; A24F 40/57; A24F 40/60; A24F 40/65; A61M 15/06; H02J 7/80

(52) U.S. Cl. CPC A24F 40/90; A24F 40/20; A24F 40/51; A24F 40/53; A24F 40/57; A24F 40/60; A24F 40/65; H02J 7/80; A61M 15/06

(57) ABSTRACT

A smoking substitute device is described along with a smoking substitute system comprising the device. The device includes a controller configured to determine the remaining usable lifetime of a consumable in a smoking session, and display said remaining usable lifetime in predetermined intervals at a display. Methods of determining the amount of remaining consumable for a session and displaying the result to the user are also described.

### CROSS REFERENCE TO RELATED APPLICATIONS/INCORPORATION BY REFERENCE STATEMENT

This application is a continuation of U.S. nonprovisional utility Ser. No. 17/481,864 , filed on 22 Sep. 2021, which is a continuation of the following patent applications: international application no. PCT/EP2020/056783, filed on 13 Mar. 2020, which claims priority to EP 19020145.9, filed on 22 Mar. 2019; international application no. PCT/EP2020/056794, filed on 13 Mar. 2020, which claims priority to EP 19020215.0, filed on 22 Mar. 2019; international application no. PCT/EP2020/056812, filed on 13 Mar. 2020, which claims priority to EP 19020146.7, filed on 22 Mar. 2019; international application no. PCT/EP2020/056795, filed on 13 Mar. 2020, which claims priority to EP 19020222.6, filed on 22 Mar. 2019; international application no. PCT/EP2020/056798, filed on 13 Mar. 2020, which claims priority to EP 19020226.7, filed on 22 Mar. 2019; international application no. PCT/EP2020/056800, filed on 13 Mar. 2020, which claims priority to EP 19020228.3, filed on 22 Mar. 2019; international application no. PCT/EP2020/056790, filed on 13 Mar. 2020, which claims priority to EP 19020162.4, filed on 22 Mar. 2019; international application no. PCT/EP2020/056817, filed on 13 Mar. 2020, which claims priority to EP 19020144.2, filed on 22 Mar. 2019; international application no. PCT/EP2020/056827, filed on 13 Mar. 2020, which claims priority to EP 19020192.1 filed on 22 Mar. 2019; international application no. PCT/EP2020/056803, filed on 13 Mar. 2020, which claims priority to EP 19020139.2, filed on 22 Mar. 2019; international application no. PCT/EP2020/056833, filed on 13 Mar. 2020, which claims priority to EP 19020198.8, filed on 22 Mar. 2019; international application no. PCT/EP2020/056801, filed on 13 Mar. 2020, which claims priority to EP 19020196.2, filed on 22 Mar. 2019; international application no. PCT/EP2020/056834, filed on 13 Mar. 2020, which claims priority to EP 19020217.6, filed on 22 Mar. 2019; international application no. PCT/EP2020/056804, filed on 13 Mar. 2020, which claims priority to EP 19020174.9 filed on 22 Mar. 2019; international application no. PCT/EP2020/056810, filed on 13 Mar. 2020, which claims priority to EP 19020202.8, filed on 22 Mar. 2019; international application no. PCT/EP2020/056835, filed on 13 Mar. 2020, which claims priority to EP 19020224.2, filed on 22 Mar. 2019; international application no. PCT/EP2020/056841, filed on 13 Mar. 2020, which claims priority to EP 19020227.5, filed on 22 Mar. 2019; and international application no. PCT/EP2020/056852, filed on 13 Mar. 2020, which claims priority to EP 19020221.8, filed on 22 Mar. 2019. The entire contents of each of the above-referenced applications are hereby incorporated herein by reference in their entirety.

### TECHNICAL FIELD

The present invention relates to a smoking substitute system and particularly, although not exclusively, to a smoking substitute system comprising a smoking substitute device and method of indicating remaining usable lifetime of a consumable in a smoking session.

### BACKGROUND

The smoking of tobacco is generally considered to expose a smoker to potentially harmful substances. It is generally thought that a significant amount of the potentially harmful substances is generated through the heat caused by the burning and/or combustion of the tobacco and the constituents of the burnt tobacco in the tobacco smoke itself.

Conventional combustible smoking articles, such as cigarettes, typically comprise a cylindrical rod of tobacco comprising shreds of tobacco which is surrounded by a wrapper, and usually also a cylindrical filter axially aligned in an abutting relationship with the wrapped tobacco rod. The filter typically comprises a filtration material which is circumscribed by a plug wrap. The wrapped tobacco rod and the filter are joined together by a wrapped band of tipping paper that circumscribes the entire length of the filter and an adjacent portion of the wrapped tobacco rod. A conventional cigarette of this type is used by lighting the end opposite to the filter and burning the tobacco rod. The smoker receives mainstream smoke into their mouth by drawing on the mouth end or filter end of the cigarette.

Combustion of organic material such as tobacco is known to produce tar and other potentially harmful by-products. There have been proposed various smoking substitute systems (or “smoking substitute systems”) in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or flavorings without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products. Some smoking substitute systems use smoking substitute articles (also referred to as a “consumables”) that are designed to resemble a traditional cigarette and are cylindrical in form with a mouthpiece at one end.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories.

There are a number of different categories of smoking substitute systems, each utilizing a different smoking substitute approach.

One approach for a smoking substitute system is the so-called Heated Tobacco (“HT”) approach in which tobacco (rather than an “e-liquid”) is heated or warmed to release vapor. HT is also known as “heat-not-burn” (“HNB”). The tobacco may be leaf tobacco or reconstituted tobacco. The vapor may contain nicotine and/or flavorings. In the HT approach the intention is that the tobacco is heated but not burned, i.e., the tobacco does not undergo combustion.

A typical HT smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes components in the tobacco material to be released as vapor. A vapor may also be formed from a carrier in the tobacco material (this carrier may for example include propylene glycol and/or vegetable glycerin) and additionally volatile compounds released from the tobacco. The released vapor may be entrained in the airflow drawn through the tobacco.

As the vapor passes through the consumable (entrained in the airflow) from the location of vaporization to an outlet of the consumable (e.g., a mouthpiece), the vapor cools and condenses to form an aerosol for inhalation by the user. The aerosol will normally contain the volatile compounds.

In HT smoking substitute systems, heating as opposed to burning the tobacco material is believed to cause fewer, or smaller quantities, of the more harmful compounds ordinarily produced during smoking. Consequently, the HT approach may reduce the odor and/or health risks that can arise through the burning, combustion, and pyrolytic degradation of tobacco.

It may be difficult for a user to determine when a consumable within a HT device needs to be replaced with a fresh consumable in order to maintain an acceptable level of volatile compounds in the aerosol stream.

There may be a need for improved design of smoking substitute systems, in particular HT smoking substitute systems, to enhance the user experience and improve the function of the HT smoking substitute system.

The present disclosure has been devised in the light of the above considerations.

### SUMMARY OF THE INVENTION

At its most general, the present invention relates to the inclusion of a controller in the smoking substitute device to determine the amount of consumable available in a session.

According to a first aspect of the present invention, there is provided a smoking substitute device for consuming a consumable in a session. The device comprises a display and a controller, the controller being configured to determine the remaining usable lifetime of the consumable, and display said remaining usable lifetime of the consumable in predetermined intervals at the display.

By providing a device comprising a controller which may be configured to determine the remaining usable lifetime of the consumable, the device has the ability to perform useful downstream functionality or provide useful information to the user. For example, one function that may be performed by the device includes determining the remaining usable lifetime of the consumable and alerting the user to the remaining usable lifetime. This may help is providing useful information to the user. Thus, an intuitive device is provided which can intelligently monitor the remaining usable lifetime of a consumable in an active smoking session.

The “remaining usable lifetime” of a consumable indicates the time remaining before the consumable is considered to be “used”, or “depleted” to such an extent that replacement with a fresh consumable is required or recommended to maintain a certain level of user experience.

“Predetermined intervals” refers to the discrete intervals of time in which the device displays the remaining usable lifetime of the consumable. A predetermined interval may be any discrete time interval and may be in terms of absolute remaining usable lifetime (e.g., in seconds, or minutes) or in terms of remaining usable lifetime relative to total usable lifetime (e.g., as a fraction, decimal or percentage). For instance, the predetermined intervals may be 25% intervals based on the total usable lifetime, such that the display shows the remaining usable lifetime of the consumable in discrete 25% units, starting with 100% and continuing in 25% intervals until 0% when the consumable is fully depleted and replacement with a fresh consumable is required.

Optional features will now be set out. These are applicable singly or in any combination with any aspect.

Optionally, the controller is configured to receive a user input and determine the remaining usable lifetime of the consumable in response to receiving user input. This gives an additional advantage to the user allowing the user to monitor the remaining usable lifetime at a time of their choosing during the session.

In some embodiments, the device comprises one or more user input means for providing user input. In one example, the user input means comprises a power button which when pressed allows the user to determine the remaining usable lifetime of the consumable. In another example, the user input means includes a sensor, wherein the sensor may be a motion sensor, audio sensor or any other like sensor that indicates the remaining consumable left upon receiving corresponding input from the user.

In some embodiments, the user input comprises one or more presses of a button on the device. When the controller detects that the button has been pressed, it determined the current usable lifetime of the consumable and displays this information on the display in a predetermined interval.

In some embodiments, the display comprises a visual representation of the remaining usable lifetime of the consumable. For example, the display may comprise a discrete number of display regions corresponding with the number of predetermined intervals, wherein the number of active display regions indicates the remaining usable lifetime. In some embodiments, the display comprises a number of LEDs, wherein the number of active (lit) LEDs corresponds with the remaining usable lifetime in predetermined intervals. For example, the display may comprise N LEDs (indicating predetermined intervals of (100/N)%), wherein the number of LEDs lit indicates the remaining usable lifetime; thus when n LEDs are lit the remaining usable lifetime shown on the display is [(100/N)×n]% of the total usable lifetime.

In some embodiments, the display regions (e.g., LEDs) become active simultaneously to indicate the remaining usable lifetime. For example, where four display regions are present to indicate remaining usable lifetime in 25% intervals, a number (e.g., two, three or four) of the display regions will become active simultaneously to indicate the remaining usable lifetime of the consumable. For example, three of the four display regions being active may indicate 75% remaining usable lifetime. Alternatively, the display regions may become active sequentially to indicate the remaining usable lifetime of the consumable. For example, two, three of four display regions (e.g., LEDs) may become active sequentially. In other embodiments, a single one of a number of display regions will become active to indicate the remaining usable lifetime of the consumable, where each individual display region is associated with a corresponding interval of remaining usable lifetime (e.g., four LEDs associated with 0%, 25%, 50% and 100% remaining usable lifetime respectively).

In some embodiments the indication of the remaining usable lifetime on the display remains active for a predetermined period of time before becoming inactive again. In some embodiments, the period of time is at least 1 second, for example at least 1.5 seconds or at least 2 seconds, to provide sufficient time for the user to observe and digest the information.

In some embodiments, the controller may be configured to determine the remaining usable lifetime of the consumable based on a pre-determined total usable lifetime. The pre-determined total usable lifetime may be stored, e.g., by a memory coupled with the controller. The pre-determined total usable lifetime may be a duration of time which is equivalent to an expected usable lifetime of a consumable. In some embodiments the device memory is pre-programmed to include this duration. The duration may be, for example, an average duration based on knowledge of the usable lifetime of consumables.

Optionally, the controller may be configured to determine the remaining usable lifetime of the consumable based on remaining time left in the smoking session. In other words, in some embodiments remaining usable lifetime is determined based on a determination of the remaining time in a smoking session, wherein the duration of a smoking session is a predetermined parameter. In some embodiments, the controller is configured to begin a smoking session when this is requested by appropriate user input, e.g., the pressing of a power button. The controller then determines the remaining time in the smoking session and displays this as the remaining usable lifetime of the consumable when requested by the user. In such embodiments, the duration of a smoking session may be predetermined based on a known period of time corresponding to, e.g., an average usable lifetime of a consumable.

In some embodiments, the controller is configured to determine the remaining usable lifetime of the consumable in proportion to total duration of the smoking session. As a result, the remaining usable lifetime may be determined and displayed as a remaining fraction, decimal or percentage of the total duration of a smoking session. For example, the display may show “75%”, indicating that 75% of the usable lifetime of the consumable remains.

In some embodiment, the controller may be configured to detect and display the remaining usable lifetime of the consumable on the display during an active smoking session. For example, the controller may be configured to indicate to the user the amount of remaining consumable during an active smoking session, i.e., when the device is switched on or the heater is being supplied with power. Thus, the user may not have to switch to any other mode to determine the amount of remaining consumable available for consumption during said active smoking session.

In some embodiments, the controller may be configured to measure ambient temperature and incorporate the ambient temperature into the determination of the remaining usable lifetime of the consumable. In some embodiments, the controller may be configured to measure the ambient temperature using a temperature sensor and calculate the amount of consumable remaining, at least partially, on the basis of detected ambient temperature.

In some embodiments, the controller may be configured to generate an alert to the user to indicate that the user is entering a final time period of usable lifetime of the consumable based on the determination of the remaining usable lifetime of the consumable. For example, if the controller has determined that the remaining usable lifetime of the consumable has fallen below a predetermined threshold, the controller may be configured to alert the user in the form of audio, video, or haptic feedback. In some embodiments, the final time period is the final 30 seconds of the usable lifetime of the consumable. In some embodiments, the controller may be configured to generate an alert to the user when user input is received requesting an indication of the remaining usable lifetime of the consumable. For example, haptic feedback (e.g., vibration of the device) may be provided to confirm to the user that the user input has been detected.

In some embodiments, the controller is configured to determine the number of puff inhalations made by the user during an active smoking session and incorporate this number into the determination of the remaining usable lifetime of the consumable. To achieve this, the controller may know the total number of puffs provided by a consumable until it is depleted and ready for replacement and monitor the number of puffs inhaled by the user in a predetermined time interval. Since the controller is aware of the amount of consumable consumed in one puff, the controller may be able to determine the amount of remaining usable lifetime at any point.

In some embodiments, the controller is configured to determine the magnitude of one or more puff inhalations made by the user during an active smoking session and incorporate this magnitude into the determination of the remaining usable lifetime of the consumable. For example, a puff of greater magnitude (e.g., a longer puff, or a puff drawn with greater force/pressure) will deplete the consumable to a greater extent than a lighter puff, and thus the total number off puffs available before the consumable is fully depleted will be smaller. A device which recognizes the magnitude of one or more puffs may therefore provide a more accurate indication of the remaining usable lifetime of the consumable than a device which merely counts the number of puffs. In some embodiments, the controller is configured to determine both the number of puff inhalations and magnitude of each puff inhalation made by the user during an active smoking session and incorporate these parameters into the determination of the remaining usable lifetime of the consumable. This provides even greater accuracy.

In some embodiments, the controller is configured to determine the type of consumable present and to incorporate this information into the determination of the remaining usable lifetime of the consumable. This will take into account any differing total usable lifetimes between different types of consumable. For example, the controller may determine that the consumable is a type with a longer total usable lifetime, adjust the length of smoking session accordingly and thereby adjust the indication of remaining usable lifetime of the consumable provided to the user. The identification of the type of consumable may be based on any suitable parameter measurable or detectable by the controller, such as a visual indicator on the surface of the consumable (e.g., a barcode), a readable medium within the consumable (e.g., a chip), measurement of the makeup of the aerosol generated by the consumable, measurement of the size/length of the consumable etc. Such a predetermined measurable parameter may be associated with a given type of consumable and the device pre-programmed accordingly.

In some embodiments, the controller is further configured, when the device is in a standby mode, to determine the remaining battery life of the device and display said battery life on the display. In some embodiments, the same display which is adapted to indicate the remaining usable lifetime of the consumable during a smoking session also indicates the remaining battery life during device standby. A standby mode may include any mode in which power is not being delivered to the heating element, e.g., before or after a smoking session. The same display or display regions may be adapted to indicate both the remaining usable lifetime of the consumable during a smoking session and the remaining battery life during device standby. Alternatively, different displays or display regions may be used.

The device may comprise an elongate body. An end of the elongate body may be configured for engagement with an aerosol-forming article. For example, the body may be configured for engagement with a heated tobacco (HT) consumable (or heat-not-burn (HNB) consumable). The terms “heated tobacco” and “heat-not-burn” are used interchangeably herein to describe a consumable that is of the type that is heated rather than combusted (or are used interchangeably to describe a device for use with such a consumable). The device may comprise a cavity that is configured for receipt of at least a portion of the consumable (i.e., for engagement with the consumable). The aerosol-forming article may be of the type that comprises an aerosol former (e.g., carried by an aerosol-forming substrate).

The device may comprise a heater for heating the aerosol-forming article. The heater may comprise a heating element, which may be in the form of a rod that extends from the body of the device. The heating element may extend from the end of the body that is configured for engagement with the aerosol-forming article.

The heater (and thus the heating element) may be rigidly mounted to the body. The heating element may be elongate so as to define a longitudinal axis and may, for example, have a transverse profile (i.e., transverse to a longitudinal axis of the heating element) that is substantially circular (i.e., the heating element may be generally cylindrical). Alternatively, the heating element may have a transverse profile that is rectangular (i.e., the heater may be a “blade heater”). The heating element may alternatively be in the shape of a tube (i.e., the heater may be a “tube heater”). The heating element may take other forms (e.g., the heating element may have an elliptical transverse profile). The shape and/or size (e.g., diameter) of the transverse profile of the heating element may be generally consistent for the entire length (or substantially the entire length) of the heating element.

The heating element may be between 15 mm and 25 mm long, e.g., between 18 mm and 20 mm long, e.g., around 19 mm long. The heating element may have a diameter of between 1.5 mm and 2.5 mm, e.g., a diameter between 2 mm and 2.3 mm, e.g., a diameter of around 2.15 mm.

The heating element may be formed of ceramic. The heating element may comprise a core (e.g., a ceramic core) comprising Al2O3. The core of the heating element may have a diameter of 1.8 mm to 2.1 mm, e.g., between 1.9 mm and 2 mm. The heating element may comprise an outer layer (e.g., an outer ceramic layer) comprising Al2O3. The thickness of the outer layer may be between 160 μm and 220 μm, e.g., between 170 μm and 190 μm, e.g., around 180 μm. The heating element may comprise a heating track, which may extend longitudinally along the heating element. The heating track may be sandwiched between the outer layer and the core of the heating element. The heating track may comprise tungsten and/or rhenium. The heating track may have a thickness of around 20 μm.

The heating element may be located in the cavity (of the device), and may extend (e.g., along a longitudinal axis) from an internal base of the cavity towards an opening of the cavity. The length of the heating element (i.e., along the longitudinal axis of the heater) may be less than the depth of the cavity. Hence, the heating element may extend for only a portion of the length of the cavity. That is, the heating element may not extend through (or beyond) the opening of the cavity.

The heating element may be configured for insertion into an aerosol-forming article (e.g., a HT consumable) when an aerosol-forming article is received in the cavity. In that respect, a distal end (i.e., distal from a base of the heating element where it is mounted to the device) of the heating element may comprise a tapered portion, which may facilitate insertion of the heating element into the aerosol-forming article. The heating element may fully penetrate an aerosol-forming article when the aerosol-forming article is received in the cavity. That is, the entire length, or substantially the entire length, of the heating element may be received in the aerosol-forming article.

The heating element may have a length that is less than, or substantially the same as, an axial length of an aerosol-forming substrate forming part of an aerosol-forming article (e.g., a HT consumable). Thus, when such an aerosol-forming article is engaged with the device, the heating element may only penetrate the aerosol-forming substrate, rather than other components of the aerosol-forming article. The heating element may penetrate the aerosol-forming substrate for substantially the entire axial length of the aerosol forming-substrate of the aerosol-forming article. Thus, heat may be transferred from (e.g., an outer circumferential surface of) the heating element to the surrounding aerosol-forming substrate, when penetrated by the heating element. That is, heat may be transferred radially outwardly (in the case of a cylindrical heating element) or, e.g., radially inwardly (in the case of a tube heater).

Where the heater is a tube heater, the heating element of the tube heater may surround at least a portion of the cavity. When the portion of the aerosol-forming article is received in the cavity, the heating element may surround a portion of the aerosol-forming article (i.e., so as to heat that portion of the aerosol-forming article). In particular, the heating element may surround an aerosol forming substrate of the aerosol-forming article. That is, when an aerosol-forming article is engaged with the device, the aerosol forming substrate of the aerosol-forming article may be located adjacent an inner surface of the (tubular) heating element. When the heating element is activated, heat may be transferred radially inwardly from the inner surface of the heating element to heat the aerosol forming substrate.

The cavity may comprise a (e.g., circumferential) wall (or walls) and the (tubular) heating element may extend around at least a portion of the wall(s). In this way, the wall may be located between the inner surface of the heating element and an outer surface of the aerosol-forming article. The wall (or walls) of the cavity may be formed from a thermally conductive material (e.g., a metal) to allow heat conduction from the heating element to the aerosol-forming article. Thus, heat may be conducted from the heating element, through the cavity wall (or walls), to the aerosol-forming substrate of an aerosol-forming article received in the cavity.

In some embodiments the device may comprise a cap disposed at the end of the body that is configured for engagement with an aerosol-forming article. Where the device comprises a heater having a heating element, the cap may at least partially enclose the heating element. The cap may be moveable between an open position in which access is provided to the heating element, and a closed position in which the cap at least partially encloses the heating element. The cap may be slidably engaged with the body of the device and may be slidable between the open and closed positions.

The cap may define at least a portion of the cavity of the device. That is, the cavity may be fully defined by the cap, or each of the cap and body may define a portion of the cavity. Where the cap fully defines the cavity, the cap may comprise an aperture for receipt of the heating element into the cavity (when the cap is in the closed position). The cap may comprise an opening to the cavity. The opening may be configured for receipt of at least a portion of an aerosol-forming article. That is, an aerosol-forming article may be inserted through the opening and into the cavity (so as to be engaged with the device).

The cap may be configured such that when an aerosol-forming article is engaged with the device (e.g., received in the cavity), only a portion of the aerosol-forming article is received in the cavity. That is, a portion of the aerosol-forming article (not received in the cavity) may protrude from (i.e., extend beyond) the opening. This (protruding) portion of the aerosol-forming article may be a terminal (e.g., mouth) end of the aerosol-forming article, which may be received in a user's mouth for the purpose of inhaling aerosol formed by the device.

The device may comprise a power source or may be connectable to a power source (e.g., a power source separate to the device). The power source may be electrically connectable to the heater. In that respect, altering (e.g., toggling) the electrical connection of the power source to the heater may affect a state of the heater. For example, toggling the electrical connection of the power source to the heater may toggle the heater between an on state and an off state. The power source may be a power store. For example, the power source may be a battery or rechargeable battery (e.g., a lithium-ion battery).

The device may comprise an input connection (e.g., a USB port, Micro USB port, USB-C port, etc.). The input connection may be configured for connection to an external source of electrical power, such as a mains electrical supply outlet. The input connection may, in some cases, be used as a substitute for an internal power source (e.g., battery or rechargeable battery). That is, the input connection may be electrically connectable to the heater (for providing power to the heater). Hence, in some forms, the input connection may form at least part of the power source of the device.

Where the power source comprises a rechargeable power source (such as a rechargeable battery), the input connection may be used to charge and recharge the power source.

The device may comprise a user interface (UI). In some embodiments the UI may include input means to receive operative commands from the user. The input means of the Ul may allow the user to control at least one aspect of the operation of the device. In some embodiments the input means may comprise a power button to switch the device between an on state and an off state. In some embodiment, the user input means may be configured to receive a user input/command to detect the amount of consumable remaining to be consumed in the session. For example, the user input means may be the power button that allows the user to determine the amount of consumable remaining in the session when pressed once or in a predetermined manner. In other example, the user input means may include a sensor, wherein the sensor may be a pressure sensor, motion sensor, audio sensor or any other like sensor.

In some embodiments the UI may additionally or alternatively comprise output means to convey information to the user. In some embodiments the output means may comprise a light to indicate a condition of the device (and/or the aerosol-forming article) to the user. The condition of the device (and/or aerosol-forming article) indicated to the user may comprise a condition indicative of the operation of the heater. For example, the condition may comprise whether the heater is in an off state or an on state. In some embodiments, the UI unit may comprise at least one of a button, a display, a touchscreen, a switch, a light, and the like. For example, the output means may comprise one or more (e.g., two, three, four, etc.) light-emitting diodes (“LEDs”) that may be located on the body of the device.

The device may further comprise a puff sensor (e.g., airflow sensor), which form part of the input means of the UI. The puff sensor may be configured to detect a user drawing on an end (i.e., a terminal (mouth) end) of the aerosol-forming article. The puff sensor may, for example, be a pressure sensor or a microphone. The puff sensor may be configured to produce a signal indicative of a puff state. The signal may be indicative of the user drawing (an aerosol from the aerosol-forming article) such that it is, e.g., in the form of a binary signal. Alternatively or additionally, the signal may be indicative of a characteristic of the draw (e.g., a flow rate of the draw, length of time of the draw, etc.).

The device may comprise a controller or may be connectable to a controller that may be configured to control at least one function of the device. The controller may comprise a microcontroller that may, e.g., be mounted on a printed circuit board (PCB). The controller may also comprise a memory, e.g., non-volatile memory. The memory may include instructions, which, when implemented, may cause the controller to perform certain tasks or steps of a method. Where the device comprises an input connection, the controller may be connected to the input connection.

The controller may be configured to control the operation of the heater (and, e.g., the heating element). Thus, the controller may be configured to control vaporization of an aerosol forming part of an aerosol-forming article engaged with the device. Further, the controller may be configured to determine the amount of remaining consumable to be consumed in a session and intimate to the user about the remaining consumable either at a predetermined interval or on user request. In addition, the controller may be configured to control the voltage applied by power source to the heater. For example, the controller may be configured to toggle between applying a full output voltage (of the power source) to the heater and applying no voltage to the heater. Alternatively or additionally, the control unit may implement a more complex heater control protocol.

The device may further comprise a voltage regulator to regulate the output voltage supplied by the power source to form a regulated voltage. The regulated voltage may subsequently be applied to the heater.

In some embodiments, where the device comprises a UI, the controller may be operatively connected to one or more components of the UI. The controller may be configured to receive command signals from an input means of the UI. The controller may be configured to control the heater in response to the command signals. For example, the controller may be configured to receive “on” and “off” command signals from the UI and, in response, may control the heater so as to be in a corresponding on or off state.

The controller may be configured to send output signals to a component of the UI. The UI may be configured to convey information to a user, via an output means, in response to such output signals (received from the controller). For example, where the device comprises one or more LEDs, the LEDs may be operatively connected to the controller. Hence, the controller may be configured to control the illumination of the LEDs (e.g., in response to an output signal). For example, the controller may be configured to control the illumination of the LEDs according to (e.g., an on or off) state of the heater.

Where the device comprises a sensor (e.g., a puff/airflow sensor), the controller may be operatively connected to the sensor. The controller may be configured to receive a signal from the sensor (e.g., indicative of a condition of the device and/or engaged aerosol-forming article). The controller may be configured to control the heater, or an aspect of the output means, based on the signal from the sensor.

The device may further include a display connected to the controller. Said display may be configured to display the amount of remaining consumable in the session, as determined by the controller. In one embodiment, the display may be configured to display the amount of remaining consumable in the session, at a predetermined interval of time. In another embodiment, the display may be configured to display the amount of remaining consumable in the session, in response to receiving user input. In an aspect, the display device may be a separate unit within the smoking substitute device or may form a part of output means of the UI.

The device may comprise a wireless interface configured to communicate wirelessly (e.g., via Bluetooth (e.g., a Bluetooth low-energy connection) or Wi-Fi) with an external device. Similarly, the input connection may be configured for wired connection to an external device so as to provide communication between the device and the external device.

The external device may be a mobile device. For example, the external device may be a smart phone, tablet, smart watch, or smart car. An application (e.g., app) may be installed on the external device (e.g., mobile device). The application may facilitate communication between the device and the external device via the wired or wireless connection.

The wireless or wired interface may be configured to transfer signals between the external device and the controller of the device. In this respect, the controller may control an aspect of the device in response to a signal received from an external device.

Alternatively or additionally, an external device may respond to a signal received from the device (e.g., from the controller of the device).

In a third aspect, there is provided a system (e.g., a smoking substitute system) comprising a device according to the first and second aspects and an aerosol-forming article. The aerosol-forming article may comprise an aerosol-forming substrate at an upstream end of the aerosol-forming article. The article may be in the form of a smoking substitute article, e.g., heated tobacco (HT) consumable (also known as a heat-not-burn (HNB) consumable).

As used herein, the terms “upstream” and “downstream” are intended to refer to the flow direction of the vapor/aerosol, i.e., with the downstream end of the article/consumable being the mouth end or outlet where the aerosol exits the consumable for inhalation by the user. The upstream end of the article/consumable is the opposing end to the downstream end.

The aerosol-forming substrate is capable of being heated to release at least one volatile compound that can form an aerosol. The aerosol-forming substrate may be located at the upstream end of the article/consumable.

In order to generate an aerosol, the aerosol-forming substrate comprises at least one volatile compound that is intended to be vaporized/aerosolized and that may provide the user with a recreational and/or medicinal effect when inhaled. Suitable chemical and/or physiologically active volatile compounds include the group consisting of nicotine, cocaine, caffeine, opiates and opioids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, Amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia califomica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

The plant material may be tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon).

The aerosol-forming substrate may comprise a gathered sheet of homogenized (e.g., paper/slurry recon) tobacco or gathered shreds/strips formed from such a sheet.

The aerosol-forming substrate may comprise one or more additives selected from humectants, flavorants, fillers, and aqueous/non-aqueous solvents and binders.

The flavorant may be provided in solid or liquid form. It may include menthol, licorice, chocolate, fruit flavor (including, e.g., citrus, cherry etc.), vanilla, spice (e.g., ginger, cinnamon) and tobacco flavor. The flavorant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm, e.g., between 6 and 9 mm or 6 and 8 mm, e.g., around 7 mm. It may have an axial length of between 10 and 15 mm, e.g., between 11 and 14 mm such as around 12 or 13 mm.

The article/consumable may comprise at least one filter element. There may be a terminal filter element at the downstream/mouth end of the article/consumable.

The or at least one of the filter element(s) (e.g., the terminal filter element) may be comprised of cellulose acetate or polypropylene tow. The at least one filter element (e.g., the terminal filter element) may be comprised of activated charcoal. The at least one filter element (e.g., the terminal element) may be comprised of paper. The or each filter element may be at least partly (e.g., entirely) circumscribed with a plug wrap, e.g., a paper plug wrap.

The terminal filter element (at the downstream end of the article/consumable) may be joined to the upstream elements forming the article/consumable by a circumscribing tipping layer, e.g., a tipping paper layer. The tipping paper may have an axial length longer than the axial length of the terminal filter element such that the tipping paper completely circumscribes the terminal filter element plus the wrapping layer surrounding any adjacent upstream element.

In some embodiments, the article/consumable may comprise an aerosol-cooling element which is adapted to cool the aerosol generated from the aerosol-forming substrate (by heat exchange) before being inhaled by the user.

The article/consumable may comprise a spacer element that defines a space or cavity between the aerosol-forming substrate and the downstream end of the consumable. The spacer element may comprise a cardboard tube. The spacer element may be circumscribed by the (paper) wrapping layer.

According to a third aspect of the present invention, there is provided a smoking substitute device configured to consume a consumable in a session. The device comprises a display and a controller, the controller being configured to determine the remaining usable lifetime of the consumable in said session based on the type of consumable, and display said remaining usable lifetime at the display.

All of the options described above in relation to the first aspect apply equally to the third aspect, mutatis mutandis.

According to a fourth aspect of the present invention, there is provided a method of using the system according to the second aspect, the method comprising inserting the aerosol-forming article into the device; and heating the article using the heater of the device.

In some embodiments the method may comprise inserting the article into a cavity within a body of the device and penetrating the article with the heating element of the device upon insertion of the article.

According to a fifth aspect of the present invention, there is provided a method of consuming a consumable in a session with a smoking substitute device comprising a display and a controller, the method comprising determining remaining usable lifetime of the consumable to be consumed in said session and displaying said remaining usable lifetime of the consumable in predetermined intervals on the display.

In some embodiments, the method comprises determining the remaining usable lifetime of the consumable in response to a user input, wherein the user input optionally comprises pressing of at least one button in a predetermined pattern for a predetermined time period.

Optionally, the method comprises measuring ambient temperature, and incorporating the ambient temperature into the determination of the remaining usable lifetime of the consumable.

In some embodiments, the method comprises determining the amount of remaining usable lifetime of the consumable based on remaining time left in the smoking session.

In some embodiments, the method comprises determining the remaining usable lifetime of the consumable in proportion to total duration of the consumable cycle.

According to a sixth aspect of the present invention, there is provided a method of consuming a consumable in a session with a smoking substitute device comprising a display and a controller, the method comprising determining the remaining usable lifetime of the consumable to be consumed in said session in response to determining type of consumable and displaying said remaining usable lifetime of the consumable on the display.

In some embodiments, the method comprises determining the remaining usable lifetime of the consumable in response to a user input, wherein the user input optionally comprises pressing of at least one button in a predetermined pattern for a predetermined time period.

Optionally, the method comprises measuring ambient temperature, and incorporating the ambient temperature into the determination of the remaining usable lifetime of the consumable.

In some embodiments, the method comprises determining the amount of remaining usable lifetime of the consumable based on remaining time left in the smoking session.

In some embodiments, the method comprises determining the remaining usable lifetime of the consumable in proportion to total duration of the consumable cycle.

Options and preferences set out above in respect of the first aspect apply equally to the fifth and sixth aspect, mutatis mutandis.

The disclosure includes the combination of the aspects and preferred features described except where such a combination is clearly impermissible or expressly avoided.

The skilled person will appreciate that except where mutually exclusive, a feature or parameter described in relation to any one of the above aspects may be applied to any other aspect. Furthermore, except where mutually exclusive, any feature or parameter described herein may be applied to any aspect and/or combined with any other feature or parameter described herein.

### SUMMARY OF THE FIGURES

So that the disclosure may be understood, and so that further aspects and features thereof may be appreciated, embodiments illustrating the principles of the disclosure will now be discussed in further detail with reference to the accompanying figures, in which:

FIG. 1A is a schematic of a smoking substitute system;

FIG. 1B is a schematic of a variation of the smoking substitute system of FIG. 1A;

FIG. 2A is a front view of a first embodiment of the smoking substitute system with the consumable engaged with the device;

FIG. 2B is a front view of the first embodiment of the smoking substitute system with the consumable disengaged from the device;

FIG. 2C is a section view of the consumable of the first embodiment of the smoking substitute system;

FIG. 2D is a detailed view of an end of the device of the first embodiment of the smoking substitute system;

FIG. 2E is a section view of the first embodiment of the smoking substitute system;

FIG. 3A is a flowchart illustrating method of determining the amount of remaining consumable in a session; and

FIG. 3B is a flowchart illustrating method of determining the amount of remaining consumable in a session, based on type of consumable.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

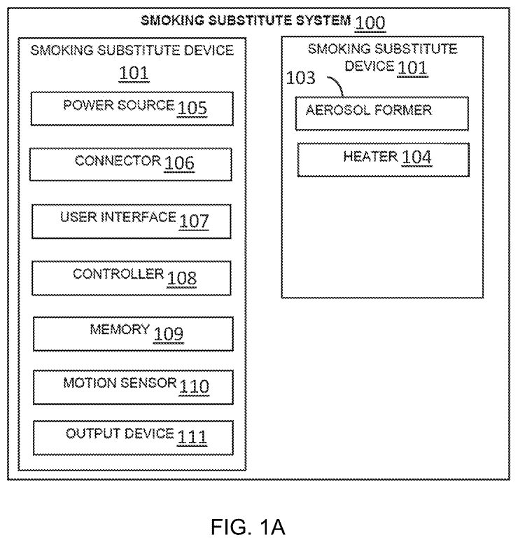

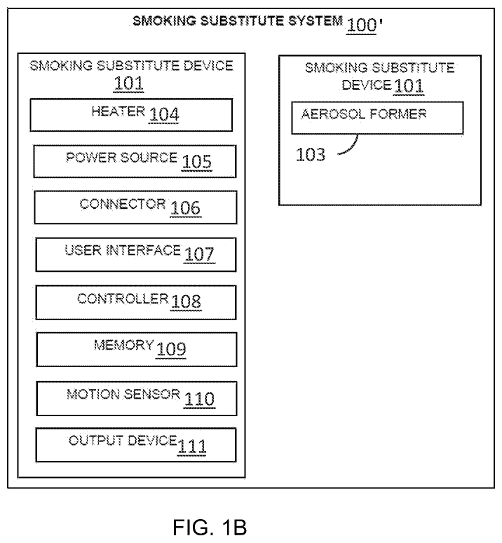

FIG. 1A is a schematic providing a general overview of a smoking substitute system 100. The system 100 includes a substitute smoking device 101 and an aerosol-forming article in the form of a consumable 102, which comprises an aerosol former 103. The system is configured to vaporize the aerosol former by heating the aerosol former 103 (so as to form a vapor/aerosol for inhalation by a user).

In the illustrated system, the heater 104 forms part of the consumable 102 and is configured to heat the aerosol former 103. Heat from the heater 104 vaporizes the aerosol former 103 to produce a vapor. The vapor subsequently condenses to form an aerosol, which is ultimately inhaled by the user.

The system 100 further comprises a power source 105 that forms part of the device 101. In other embodiments the power source 105 may be external to (but connectable to) the device 101. The power source 105 is electrically connectable to the heater 104 such that the power source 105 is able to supply power to the heater 104 (i.e., for the purpose of heating the aerosol former 103). Thus, control of the electrical connection of the power source 105 to the heater 104 provides control of the state of the heater 104. The power source 105 may be a power store, for example a battery or rechargeable battery (e.g., a lithium-ion battery).

The system 100 further comprises an I/O module comprising a connector 106 (e.g., in the form of a USB port, Micro USB port, USB-C port, etc.). The connector 106 is configured for connection to an external source of electrical power, e.g., a mains electrical supply outlet. The connector 106 may be used in substitution for the power source 105. That is the connector 106 may be electrically connectable to the heater 104 so as to supply electricity to the heater 104. In such embodiments, the device may not include a power source, and the power source of the system may instead comprise the connector 106 and an external source of electrical power (to which the connector 106 provides electrical connection).

In some embodiments, the connector 106 may be used to charge and recharge the power source 105 where the power source 105 includes a rechargeable battery.

The system 100 also comprises a user interface (UI) 107. Although not shown, the UI 107 may include input means to receive commands from a user. The input means of the UI 107 allows the user to control at least one aspect of the operation of the system 100. The input means may, for example, be in the form of a button, touchscreen, switch, microphone, etc.

The UI 107 also comprises output means to convey information to the user. The output means may, for example, comprise lights (e.g., LEDs), a display screen, speaker, vibration generator, etc. The system 100 may further comprise a display 111. The display 111 may be configured to display to the user the remaining usable lifetime of the consumable. In one embodiment, the display screen of the UI 107 may act as display 111. In other embodiment, the display 111 may be a separate unit, i.e., outside the UI 107, as depicted in FIG. 1A and FIG. 1B.

The system 100 further comprises a controller 108 and a memory 109 operatively coupled to the controller 108 that is configured to control at least one function of the device 101. In the illustrated embodiment, the controller 108 is a component of the device 101, but in other embodiments may be separate from (but connectable to) the device 101. The controller 108 may be configured to determine the remaining usable lifetime of a consumable to be consumed in a session. The controller 108 may be configured to determine the remaining usable lifetime of a consumable upon receiving user request. In addition, the controller 108 may be configured to determine the type of consumable 103 used with the device 101 and determine the remaining usable lifetime of a consumable based on type of consumable detected. The controller 108 is further configured to control the operation of the heater 104 and, for example, may be configured to control the voltage applied from the power source 105 to the heater 104. The controller 108 may be configured to toggle the supply of power to the heater 104 between an on state, in which the full output voltage of the power source 105 is applied to the heater 104, and an off state, in which the no voltage is applied to the heater 104.

Although not shown, the system 100 may also comprise a voltage regulator to regulate the output voltage from the power source 105 to form a regulated voltage. The regulated voltage may then be applied to the heater 104.

In addition to being connected to the heater 104, the controller 108 is operatively connected to the UI 107. Thus, the controller 108 may receive an input signal from the input means of the UI 107. Similarly, the controller 108 may transmit output signals to the UI 107. In response, the output means of the UI 107 may convey information, based on the output signals, to a user.

Further, the system 100 may comprise a sensor 110 coupled with the controller 108 within the device 101. The sensor 110 may be a puff sensor mounted inside the device 101 and configured to keep a count of number and/or magnitude of puffs inhaled during a session. Further, the sensor 110 may be a temperature sensor configured to determine the ambient temperature.

FIG. 1B is a schematic showing a variation of the system 100 of FIG. 1A. In the system 100′ of FIG. 1B, the heater 104 forms part of the device 101, rather than the consumable 102. In this variation, the heater 104 is electrically connected to the power source 105.

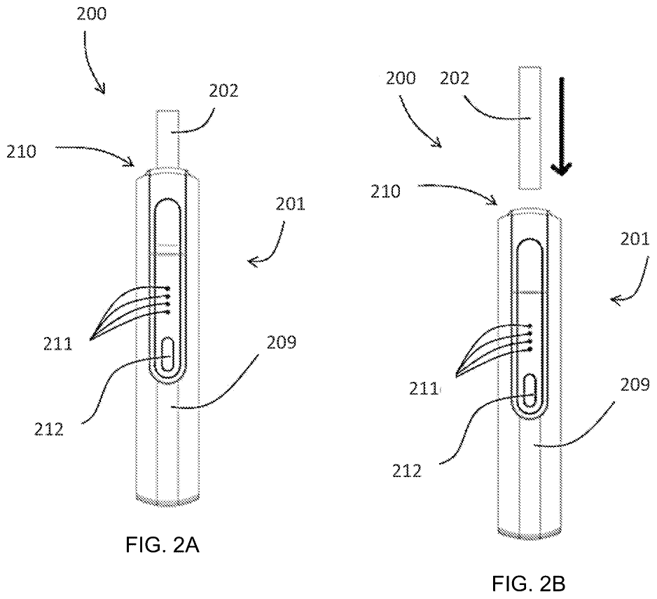

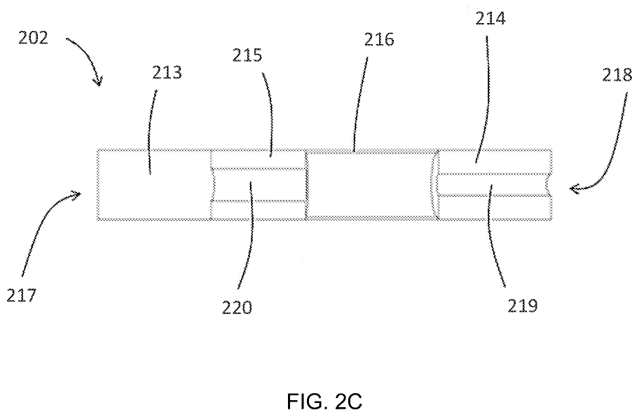

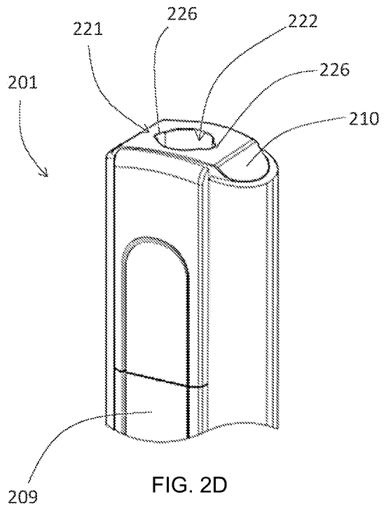

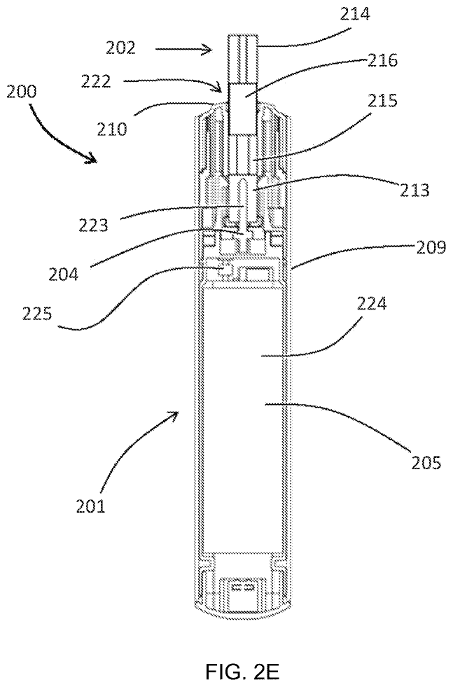

FIG. 2A and FIG. 2B illustrate a heated-tobacco (HT) smoking substitute system 200. The system 200 is an example of the systems 100, 100′ described in relation to FIG. 1A or FIG. 1B. System 200 includes an HT device 201 that is configured to determine the remaining usable lifetime of a consumable 202 left for consumption in a session and an HT consumable 202. The description of FIG. 1A and FIG. 1B above is applicable to the system 200 of FIG. 2A and FIG. 2B and will not be repeated.

The device 201 and the consumable 202 are configured such that the consumable 202 can be engaged with the device 201. FIG. 2A shows the device 201 and the consumable 202 in an engaged state, whilst FIG. 2B shows the device 201 and the consumable 202 in a disengaged state.

The device 201 comprises a body 209 and cap 210. In use the cap 210 is engaged at an end of the body 209. Although not apparent from the figures, the cap 210 is moveable relative to the body 209. In particular, the cap 210 is slidable and can slide along a longitudinal axis of the body 209.

The device 201 comprises an output means (forming part of the UI of the device 201) in the form of a plurality of light-emitting diodes (LEDs) 211 arranged linearly along the longitudinal axis of the device 201 and on an outer surface of the body 209 of the device 201. A button 212 is also arranged on an outer surface of the body 209 of the device 201 and is axially spaced (i.e., along the longitudinal axis) from the plurality of LEDs 211.

FIG. 2C show a detailed section view of the consumable 202 of the system 200. The consumable 202 generally resembles a cigarette. In that respect, the consumable 202 has a generally cylindrical form with a diameter of 7 mm and an axial length of 70 mm. The consumable 202 comprises an aerosol forming substrate 213, a terminal filter element 214, an upstream filter element 215 and a spacer element 216. In other embodiments, the consumable may further comprise a cooling element. A cooling element may exchange heat with vapor that is formed by the aerosol-forming substrate 213 in order to cool the vapor so as to facilitate condensation of the vapor.

The aerosol-forming substrate 213 is substantially cylindrical and is located at an upstream end 217 of the consumable 202 and comprises the aerosol former of the system 200. In that respect, the aerosol forming substrate 213 is configured to be heated by the device 201 to release a vapor. The released vapor is subsequently entrained in an airflow flowing through the aerosol-forming substrate 213. The airflow is produced by the action of the user drawing on a downstream end 218 (i.e., terminal or mouth end) of the consumable 202.

In the present embodiment, the aerosol forming substrate 213 comprises tobacco material that may, for example, include any suitable parts of the tobacco plant (e.g., leaves, stems, roots, bark, seeds, and flowers). The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon). For example, the aerosol-forming substrate 213 may comprise a gathered sheet of homogenized (e.g., paper/slurry recon) tobacco or gathered shreds/strips formed from such a sheet.

In order to generate an aerosol, the aerosol forming substrate 213 comprises at least one volatile compound that is intended to be vaporized/aerosolized and that may provide the user with a recreational and/or medicinal effect when inhaled. The aerosol-forming substrate 213 may further comprise one or more additives. For example, such additives may be in the form of humectants (e.g., propylene glycol and/or vegetable glycerin), flavorants, fillers, aqueous/non-aqueous solvents and/or binders.

The terminal filter element 214 is also substantially cylindrical and is located downstream of the aerosol forming substrate 213 at the downstream end 218 of the consumable 202. The terminal filter element 214 is in the form of a hollow bore filter element having a bore 219 (e.g., for airflow) formed therethrough. The diameter of the bore 219 is 2 mm. The terminal filter element 214 is formed of a porous (e.g., monoacetate) filter material. As set forth above, the downstream end 218 of the consumable 202 (i.e., where the terminal filter 214 is located) forms a mouthpiece portion of the consumable 202 upon which the user draws. Airflow is drawn from the upstream end 217, thorough the components of the consumable 202, and out of the downstream end 218. The airflow is driven by the user drawing on the downstream end 218 (i.e., the mouthpiece portion) of the consumable 202.

The upstream filter element 215 is located axially adjacent to the aerosol-forming substrate 213, between the aerosol-forming substrate 213 and the terminal filter element 214. Like the terminal filter 214, the upstream filter element 215 is in the form of a hollow bore filter element, such that it has a bore 220 extending axially therethrough. In this way, the upstream filter 215 may act as an airflow restrictor. The upstream filter element 215 is formed of a porous (e.g., monoacetate) filter material. The bore 220 of the upstream filter element 215 has a larger diameter (3 mm) than the terminal filter element 214.

The spacer 216 is in the form of a cardboard tube, which defines a cavity or chamber between the upstream filter element 215 and the terminal filter element 214. The spacer 216 acts to allow both cooling and mixing of the vapor/aerosol from the aerosol-forming substrate 213. The spacer has an external diameter of 7 mm and an axial length of 14 mm.

Although not apparent from the figure, the aerosol-forming substrate 213, upstream filter 215 and spacer 216 are circumscribed by a paper wrapping layer. The terminal filter 214 is circumscribed by a tipping layer that also circumscribes a portion of the paper wrapping layer (so as to connect the terminal filter 214 to the remaining components of the consumable 202). The upstream filter 215 and terminal filter 214 are circumscribed by further wrapping layers in the form of plug wraps.

Returning now to the device 201, FIG. 2D illustrates a detailed view of the end of the device 201 that is configured to engage with the consumable 202. The cap 210 of the device 201 includes an opening 221 to an internal cavity 222 (more apparent from FIG. 2D) defined by the cap 210. The opening 221 and the cavity 222 are formed so as to receive at least a portion of the consumable 202. During engagement of the consumable 202 with the device 201, a portion of the consumable 202 is received through the opening 221 and into the cavity 222. After engagement (see FIG. 2B, the downstream end 218 of the consumable 202 protrudes from the opening 221 and thus also protrudes from the device 201. The opening 221 includes laterally disposed notches 226. When a consumable 202 is received in the opening 221, these notches 226 remain open and could, for example, be used for retaining a cover in order to cover the end of the device 201.

FIG. 2E shows a cross section through a central longitudinal plane through the device 201. The device 201 is shown with the consumable 202 engaged therewith.

The device 201 comprises a heater 204 comprising heating element 223. The heater 204 forms part of the body 209 of the device 201 and is rigidly mounted to the body 209. In the illustrated embodiment, the heater 204 is a rod heater with a heating element 223 having a circular transverse profile. In other embodiments the heater may be in the form of a blade heater (e.g., heating element with a rectangular transverse profile) or a tube heater (e.g., heating element with a tubular form).

The heating element 223 of the heater 204 projects from an internal base of the cavity 222 along a longitudinal axis towards the opening 221. As is apparent from the figure, the length (i.e., along the longitudinal axis) of the heating element is less than a depth of the cavity 222. In this way, the heating element 223 does not protrude from or extend beyond the opening 221.

When the consumable 202 is received in the cavity 222 (as is shown in FIG. 2E), the heating element 223 penetrates the aerosol-forming substrate 213 of the consumable 202. In particular, the heating element 223 extends for nearly the entire axial length of the aerosol-forming substrate 213 when inserted therein. Thus, when the heater 204 is activated, heat is transferred radially from an outer circumferential surface the heating element 223 to the aerosol-forming substrate 213.

The device 201 further comprises an electronics cavity 224. A power source, in the form of a rechargeable battery 205 (a lithium-ion battery), is located in electronics cavity 224.

The device 201 includes a connector (i.e., forming part of an IO module of the device 201) in the form of a USB port 206. The connector may alternatively be, for example, a micro-USB port or a USB-C port for examples. The USB port 206 may be used to recharge the rechargeable battery 205.

The device 201 includes a controller (not shown) located in the electronics cavity 224. The controller comprises a microcontroller mounted on a printed circuit board (PCB). The USB port 206 is also connected to the controller 208 (i.e., connected to the PCB and microcontroller).

The controller 208 is configured to control at least one function of the device 201. For example, the controller 208 is configured to control the operation of the heater 204. Such control of the operation of the heater 204 may be accomplished by the controller toggling the electrical connection of the rechargeable battery 205 to the heater 204. For example, the controller 208 is configured to control the heater 204 in response to a user depressing the button 212. Depressing the button 212 may cause the controller to allow a voltage (from the rechargeable battery 205) to be applied to the heater 204 (so as to cause the heating element 223 to be heated).

In an aspect, the controller 208 may be configured to determine the remaining usable lifetime of a consumable 202 to be consumed in a session. The controller 208 may be configured to determine the remaining usable lifetime of a consumable 202 in response to receiving user request/input. The controller 208 may be configured to receive the user input/request via one of the input means of the UI 107. For example, the controller 208 may be configured to determine the remaining usable lifetime in response to the user pressing the power button or a separate, bespoke usable lifetime status button.

In one aspect, to determine the remaining usable lifetime of a consumable 202, the controller 208 may use remaining time left in the smoking session. To achieve this the controller 208 may be configured to provide a smoking session of a duration corresponding with the expected total lifetime of a consumable 202. Simultaneously, the controller 208 may also keep a track of time elapsed during said session in which the consumable 202 was consumed. Based on this information the controller 208 may be configured to calculate the remaining usable lifetime of the consumable at any given point of time and present this information to the user in discrete intervals on the display.

In other aspect, to determine the remaining usable lifetime of the consumable 202, the controller 208 may make use of total duration of the smoking session. To achieve this the controller 208 may be configured to first determine the total duration of the smoking session. The controller 208 may be further configured to track elapsed time in the smoking session. Based on said information the controller 208 may be configured to determine the remaining usable lifetime of the consumable.

In another aspect, the controller 208 may be configured to measure ambient temperature and incorporate the ambient temperature into the determination of the remaining usable lifetime of the consumable 202. To achieve this, the controller 208 may be configured to measure the ambient temperature using the sensor 110. The calculation to determine the amount of remaining usable lifetime of the consumable 202 may be based on the relationship of the consumable 202 with the ambient temperature, i.e., amount of consumable 202 that may get vaporized/used on a certain ambient temperature.

In another aspect, the controller 208 may make use of the number and/or magnitude of puffs inhaled by the user. In an example, the controller 208 may be configured to determine the remaining usable lifetime of the consumable 202 based on the number of puffs inhaled by the user. For this, the controller may keep a track of total puffs available during a session and the number of puffs inhaled by the user. Since, the controller 208 is aware of the amount of consumable consumed in one puff, the controller 208 may be able to determine the remaining usable lifetime.

To make this possible, the device 201 may comprise a puff sensor 225 connected to the controller 208 and configured to detect at least one puff inhaled by the user during a session. In one embodiment, the controller 208 may be configured to keep a track of each puff inhaled by the user and detected by the sensor 225 during said session. The puff sensor 225 is configured to pass on information to the controller 208 when a puff is detected, which may include (a) that a puff has been detected, and/or (b) the magnitude of that puff. The controller 208 uses said information to determine the remaining usable lifetime.

In another aspect, the controller 208 may be configured to determine the remaining usable lifetime based on the type of consumable 202. The controller 208 may have prestored information about different type of consumable 202 that may be used in conjunction with the device 201. In one example, the controller 208 may have information regarding each type of consumable 202 and the consumable cycle for each of these consumables 202. Thus, the controller 208 first needs to determine the type of consumable 202 inserted in the device 201, which may be detected by a sensor (not shown). The controller 208 may than use the prestored information to determine the remaining usable lifetime at a given point during the session.

Further, the controller 208 is connected to the display 111. Said display 111 may be configured to display the remaining usable lifetime, as determined by the controller 208. The display 111 may be configured to display the remaining usable lifetime in response to receiving user input/request, e.g., pressing of a button on the UI.

In some embodiment, the controller 208 may be configured to determine and display the remaining usable lifetime during an active smoking session, i.e., when the heater 204 of the device 201 is switched on. In some embodiments, the controller 208 may be configured to generate an alert to the user to indicate that the user has requested information on the remaining usable lifetime or is entering the final time period of the usable lifetime based on the determination.

In one example, to provide an alert the device 201 may include a haptic feedback device (not shown) connected to the controller 208. The haptic feedback device may be configured to provide haptic feedback to the user.

The controller 208 is also configured to control the LEDs 211 in response to (e.g., a detected) a condition of the device 201 or the consumable 202. For example, the controller may control the LEDs to indicate whether the device 201 is in an on state or an off state (e.g., one or more of the LEDs may be illuminated by the controller when the device is in an on state).

In an embodiment of the disclosure, the controller 208 is configured to determine the remaining usable lifetime of the consumable in said session, and display said remaining usable lifetime by means of the LEDs 211. The four LEDs 211 indicate the remaining usable lifetime of the consumable in 25% intervals. When the user presses the button 212 during an active smoking session, a certain number of the LEDs 211 light up depending on the remaining usable lifetime of the consumable as determined by the controller and remain lit for 2 seconds. If the controller determines that the remaining usable lifetime of the consumable is greater than 75%, all four LEDs light up. If the controller determines that the remaining usable lifetime of the consumable is greater than 50% and up to and including 75%, three of the four LEDs 211 light up. If the controller determines that the remaining usable lifetime of the consumable is greater than 25% and up to and including 50%, two of the four LEDs light up. If the controller determines that the remaining usable lifetime of the consumable is greater than 0% and up to and including 25%, one of the four LEDs light up. If the controller determines that the remaining usable lifetime of the consumable is 0% (i.e., the consumable is fully depleted), none of the LEDs light up and the user becomes aware that replacement of the consumable is recommended to maintain a good user experience.

Moving upwards in FIG. 2A, the four LEDs 211 may be denoted 211-1, 211-2, 211-3, and 211-4 (not shown in the Figure). In another embodiment of the invention, when the user presses the button 212 during an active smoking session, a single one of the LEDs 211 lights up for 2 seconds, and the particular LED which lights up depends on the remaining usable lifetime of the consumable as determined by the controller. If the controller determines that the remaining usable lifetime of the consumable is greater than 75%, LED 211-4 lights up. If the controller determines that the remaining usable lifetime of the consumable is greater than 50% and up to and including 75%, LED 211-3 lights up. If the controller determines that the remaining usable lifetime of the consumable is greater than 25% and up to and including 50%, LED 211-2 lights up. If the controller determines that the remaining usable lifetime of the consumable is greater than 0% and up to and including 25%, LED 211-1 lights up. If the controller determines that the remaining usable lifetime of the consumable is 0% (i.e., the consumable is fully depleted), none of the LEDs light up and the user becomes aware that replacement of the consumable is recommended to maintain a good user experience. In this embodiment, each of the preceding LEDs in the sequence 211-1, 211-2, 211-3, and 211-4 may light up sequentially before the final indicative LED lights up. For example, if the controller determines that the remaining usable lifetime of the consumable is greater than 50% and up to and including 75%, LED 211-1, LED 211-2 and LED 211-3 light up sequentially, with LED 211-3 then remaining lit for 2 seconds.

Although not shown in the Figures, there may be an indicator on the display beside the LEDs 211 reflecting the remaining usable lifetime of the consumable indicated when that LED lights up. For example, the indicator “75%” may be printed on the device beside LED 211-3.

When the device 201 is in standby mode, the controller 208 is configured instead to display the remaining battery life of the device 201 using the LEDs 211. In one embodiment of the invention, when the device is in standby mode and the user presses the button 212, if the controller determines that the remaining battery life is greater than 75%, all four LEDs light up. If the controller determines that the remaining battery life is 0% (i.e., the battery is fully depleted), none of the LEDs light up and the user becomes aware that recharging of the device is required.

The device 201 comprises a further input means (i.e., in addition to the button 212) in the form of the puff sensor 225. As discussed, the puff sensor 225 is configured to detect a user drawing (i.e., inhaling) at the downstream end 218 of the consumable 202. The puff sensor 225 may, for example, be in the form of a pressure sensor, a flowmeter, or a microphone. The puff sensor 225 is operatively connected to the controller 208 in the electronics cavity 224, such that a signal from the puff sensor 225, indicative of a puff state (i.e., drawing or not drawing), forms an input to the controller 208 (and can thus be responded to by the controller 208).

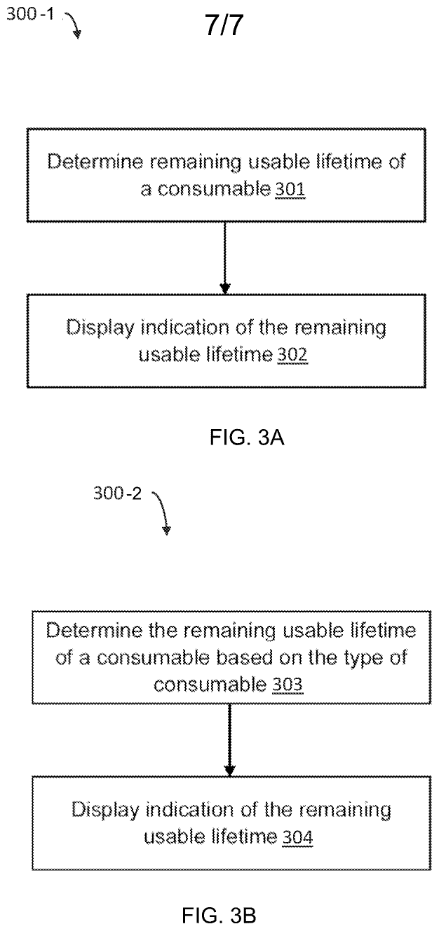

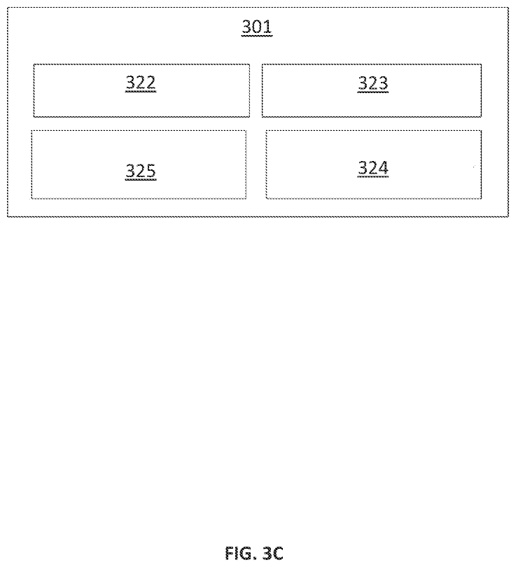

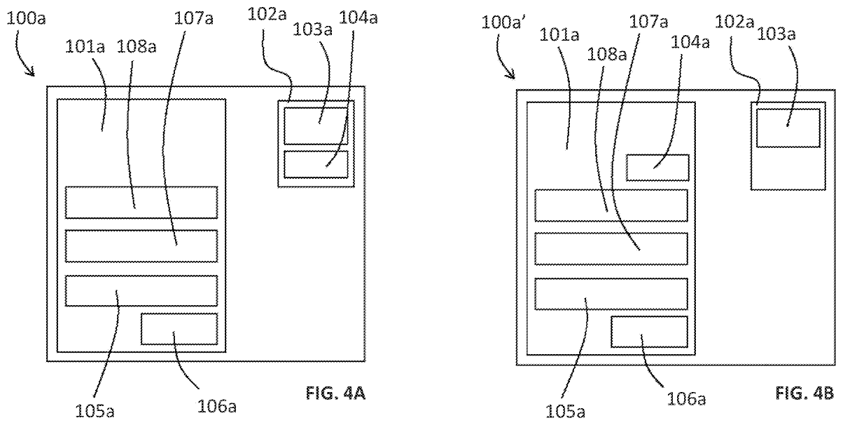

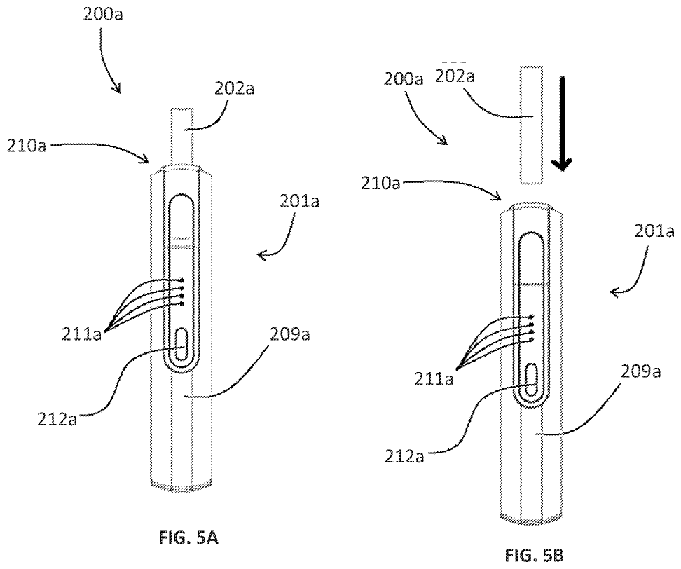

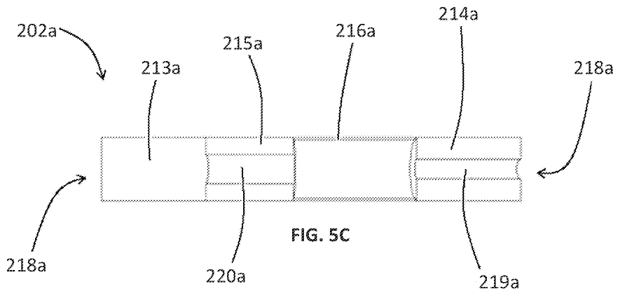

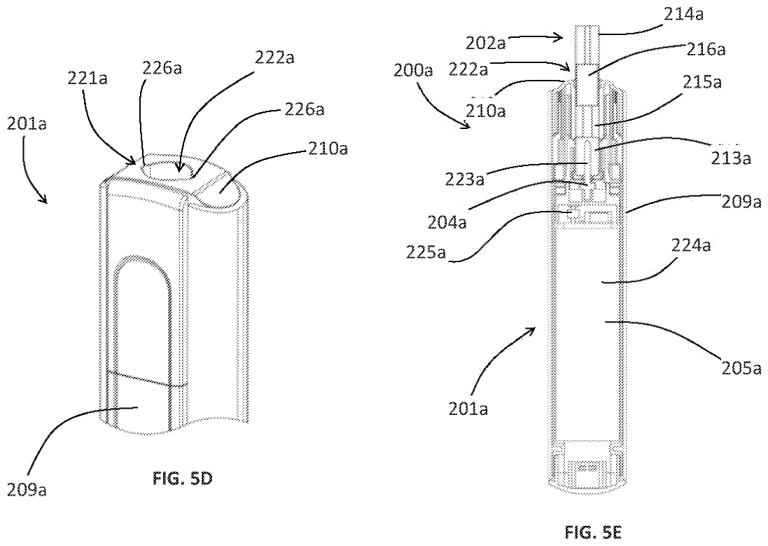

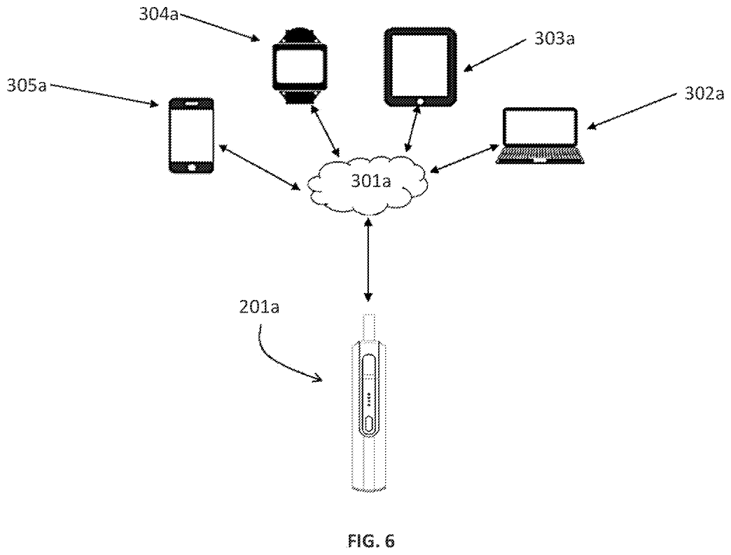

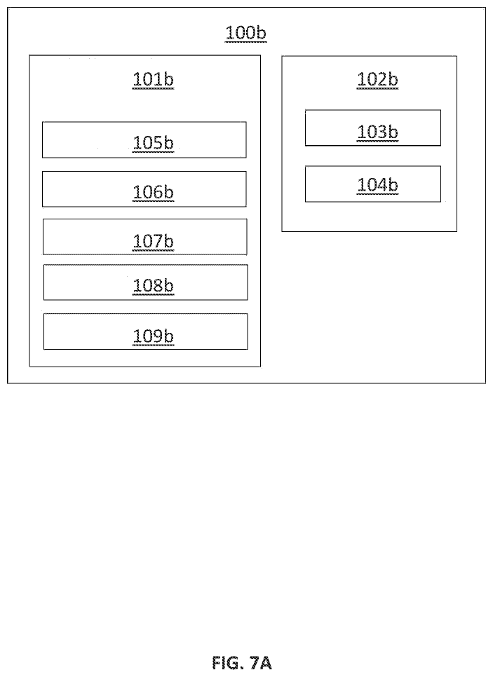

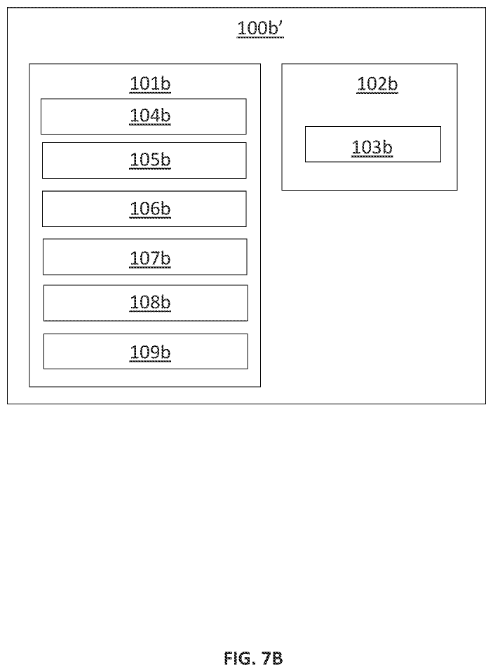

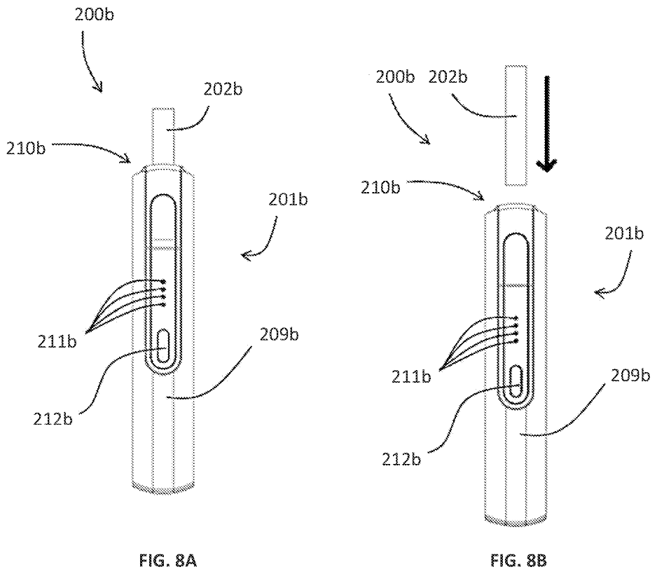

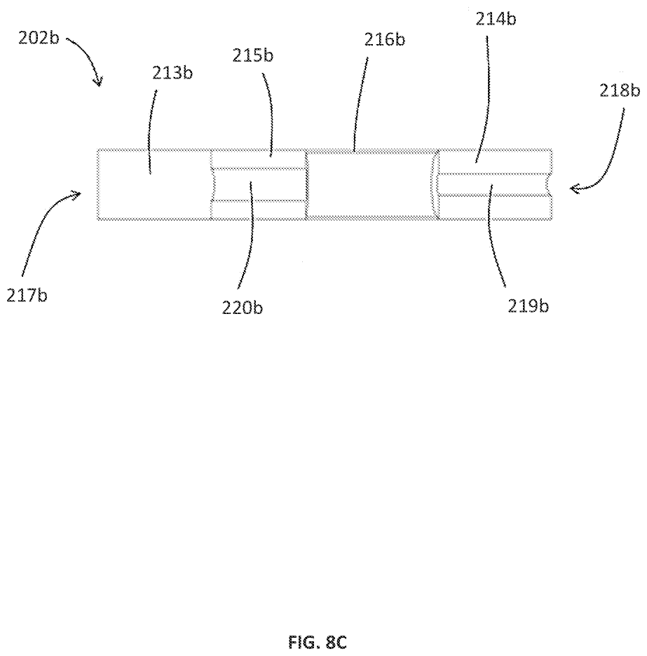

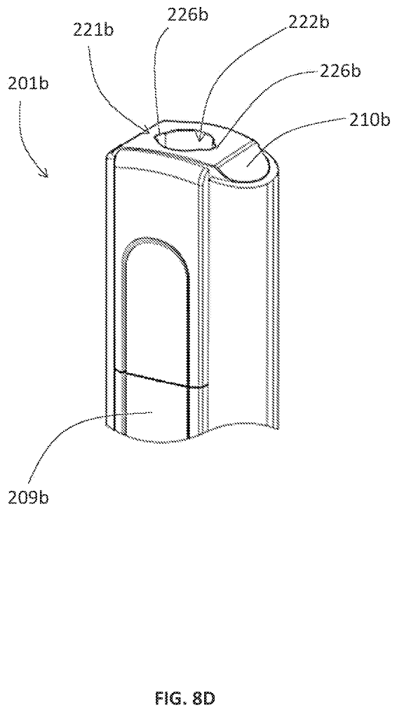

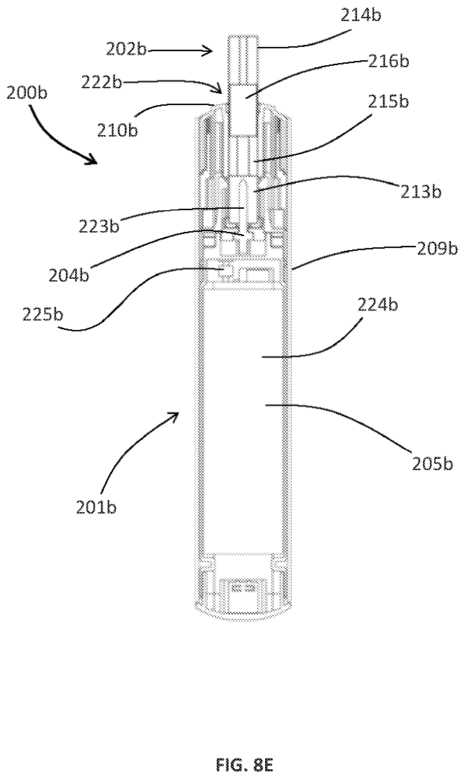

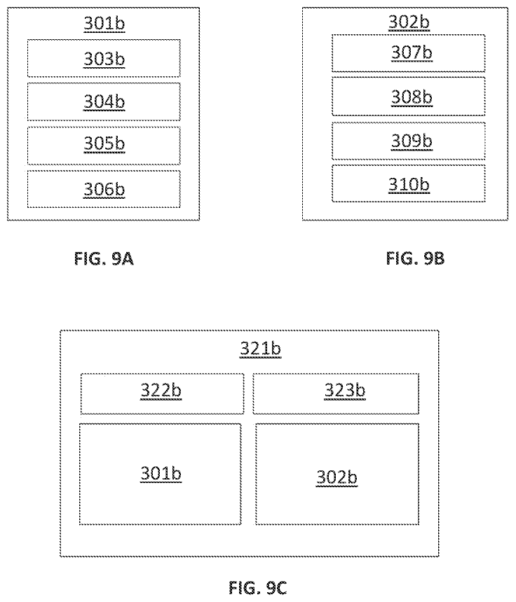

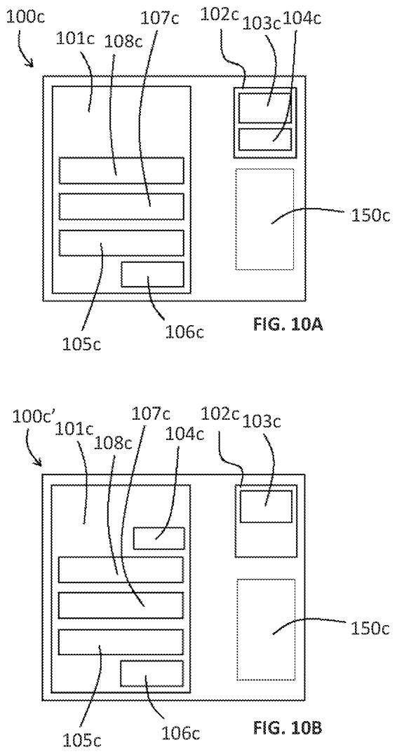

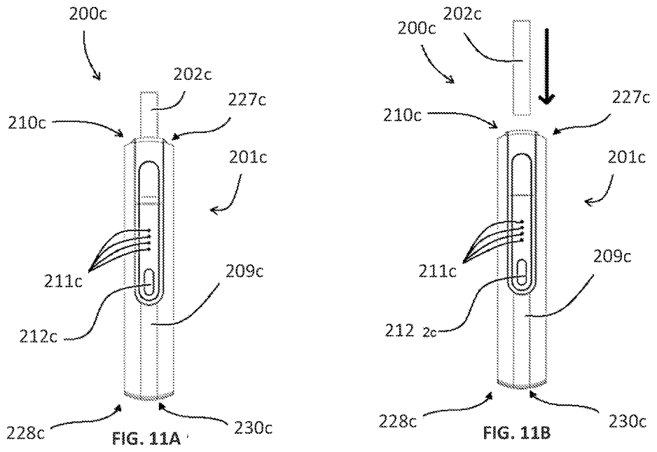

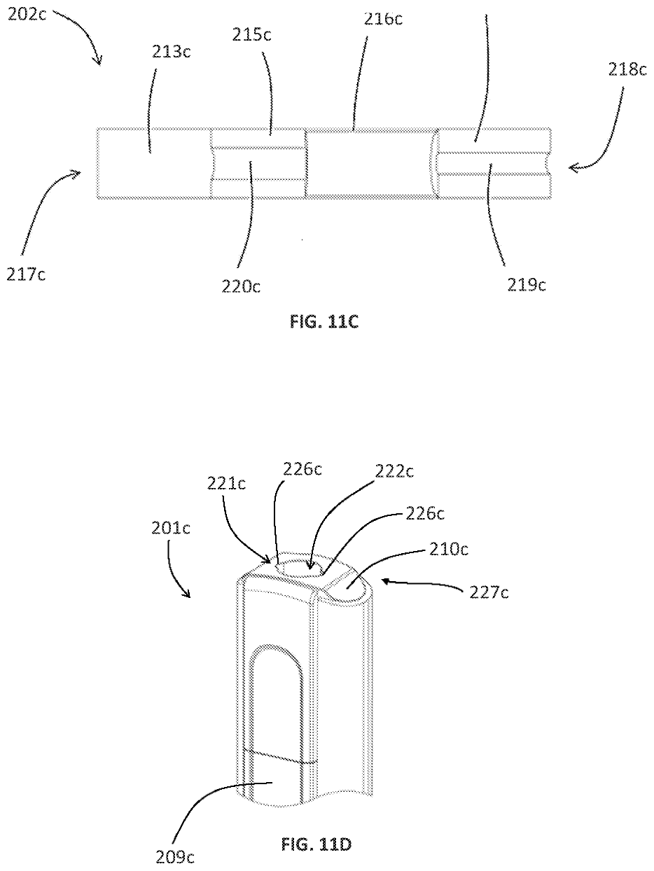

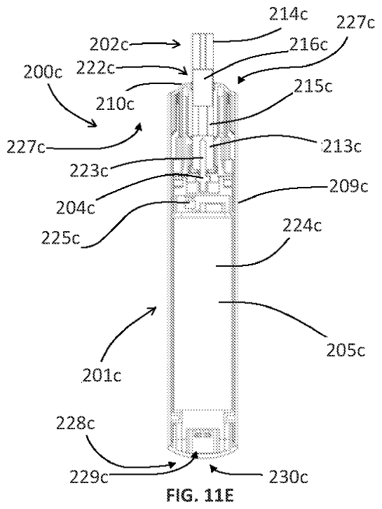

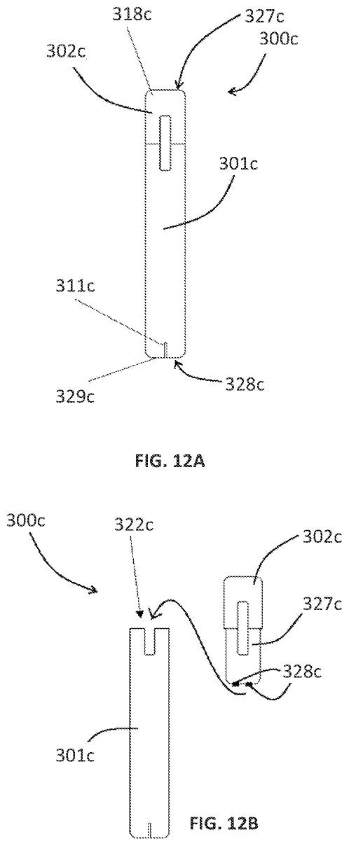

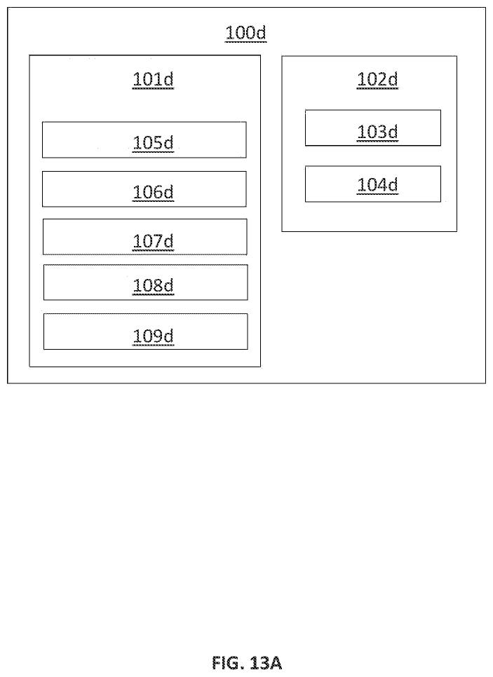

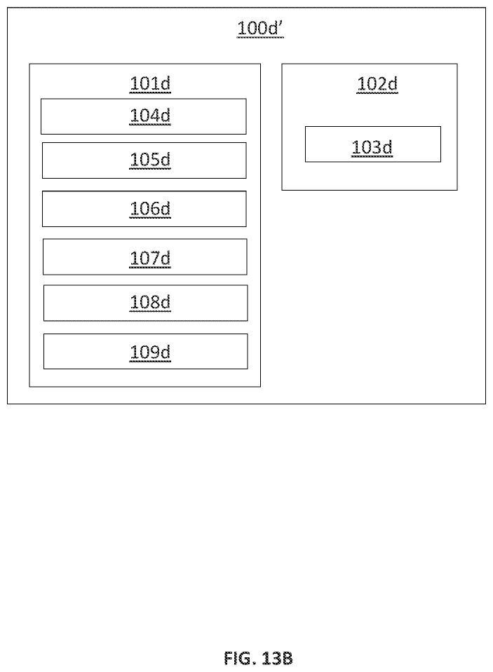

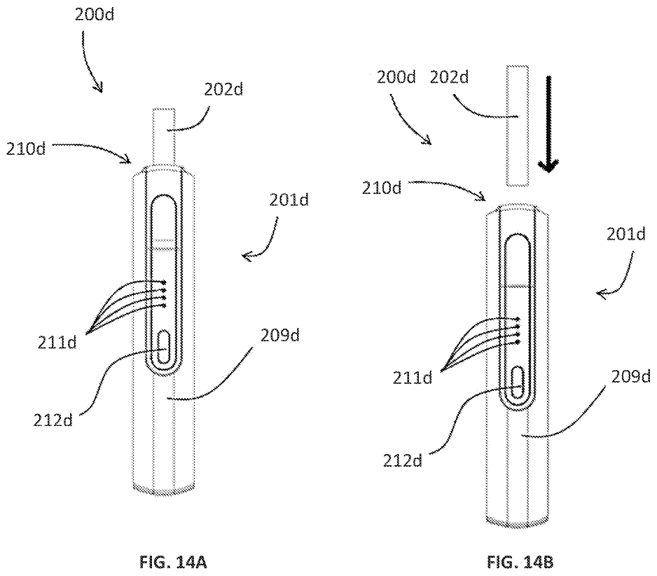

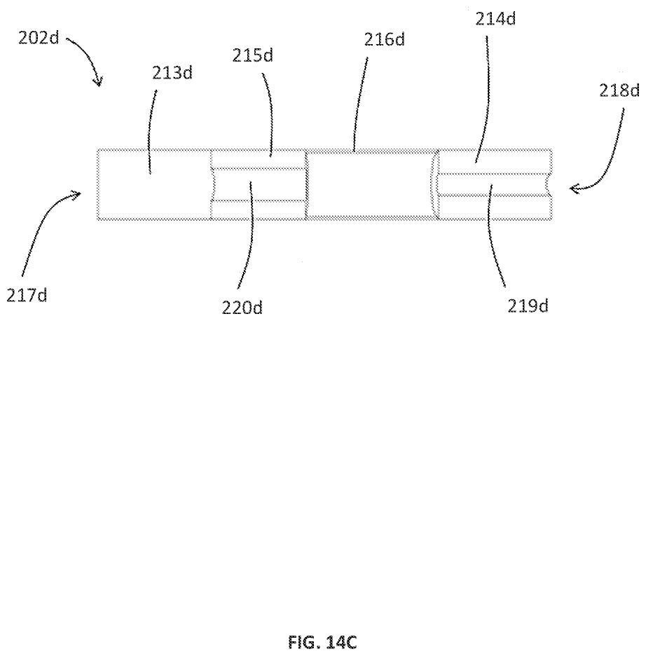

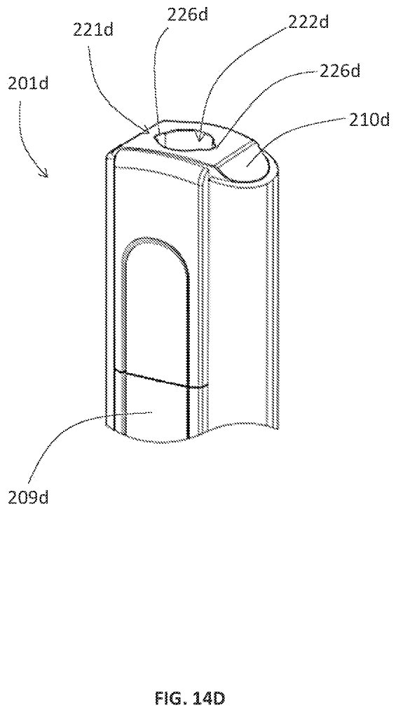

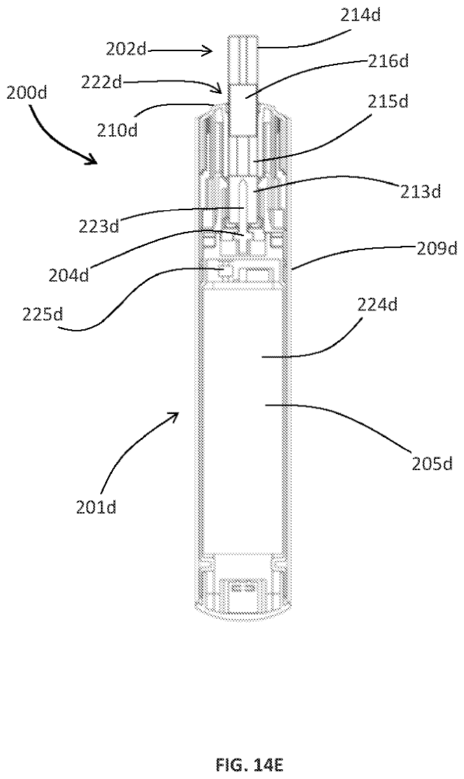

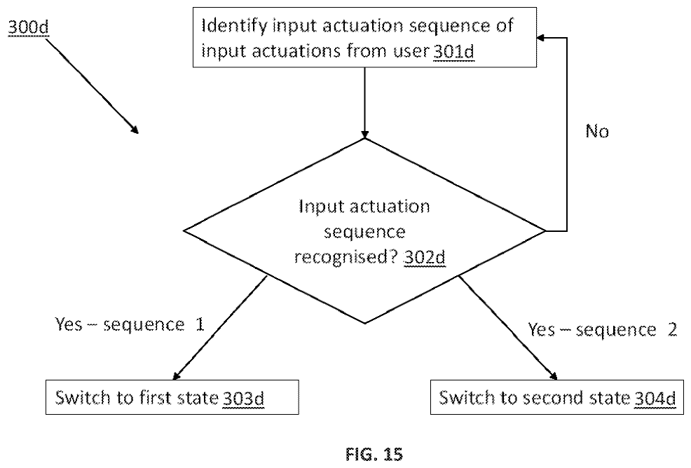

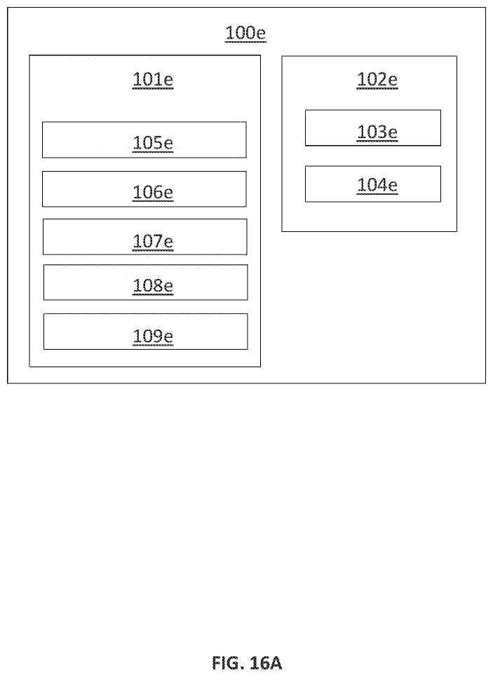

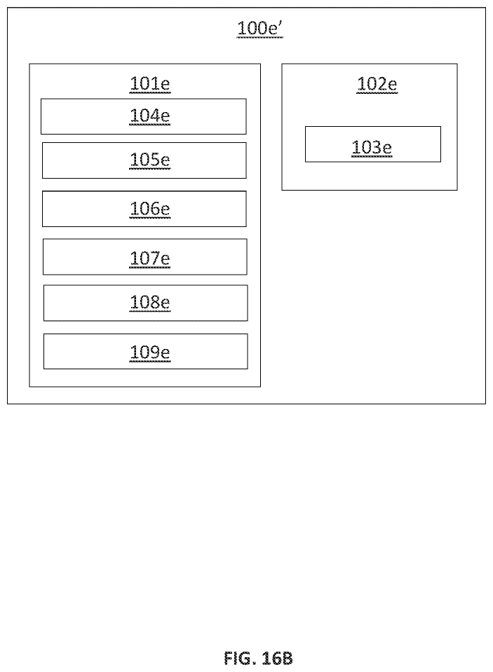

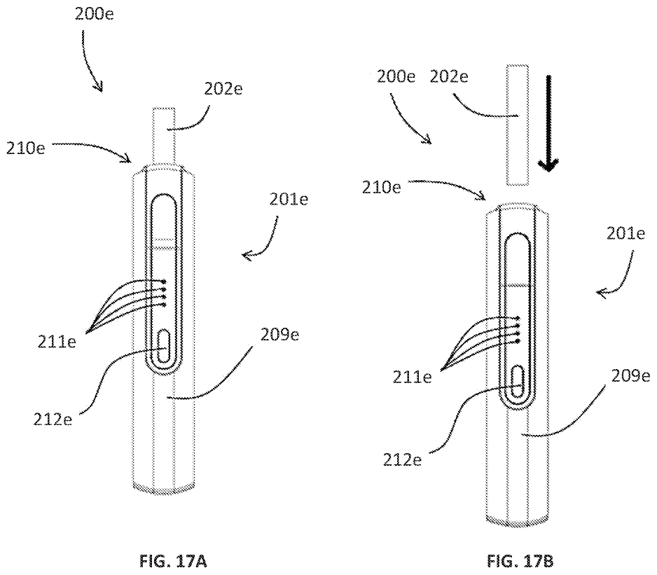

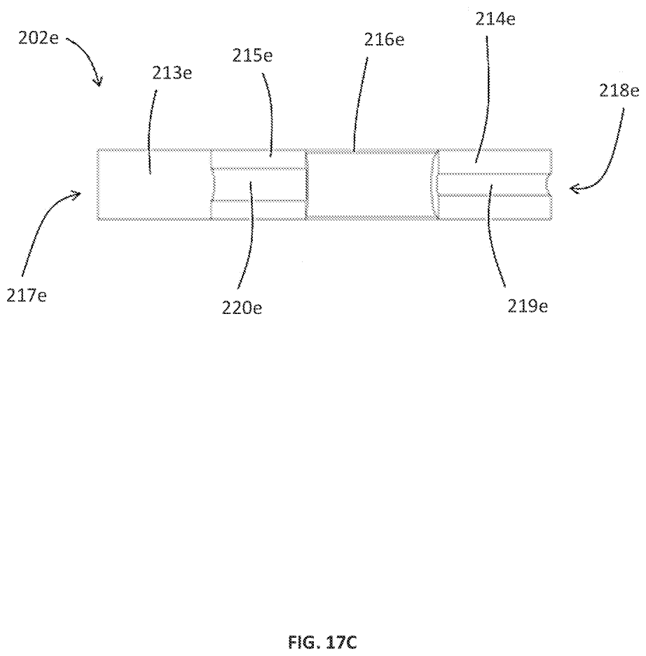

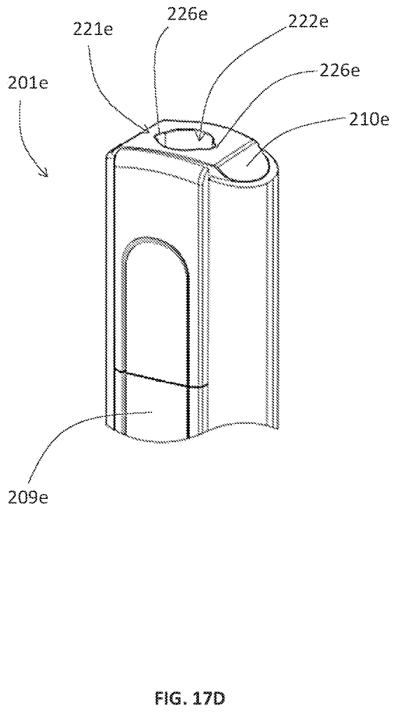

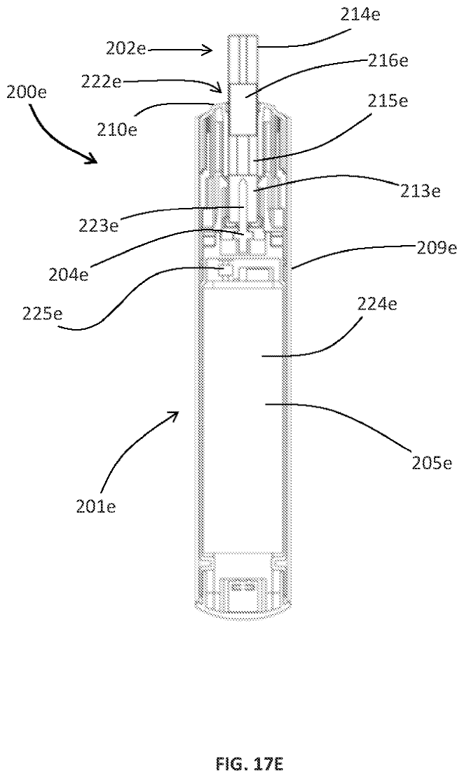

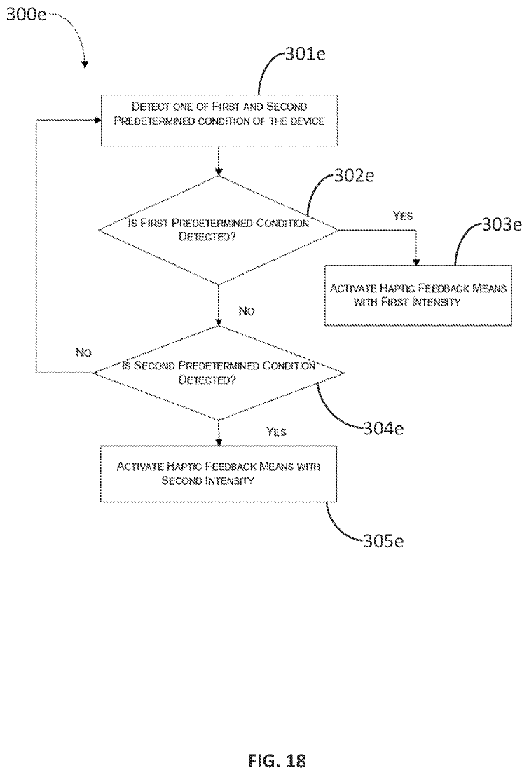

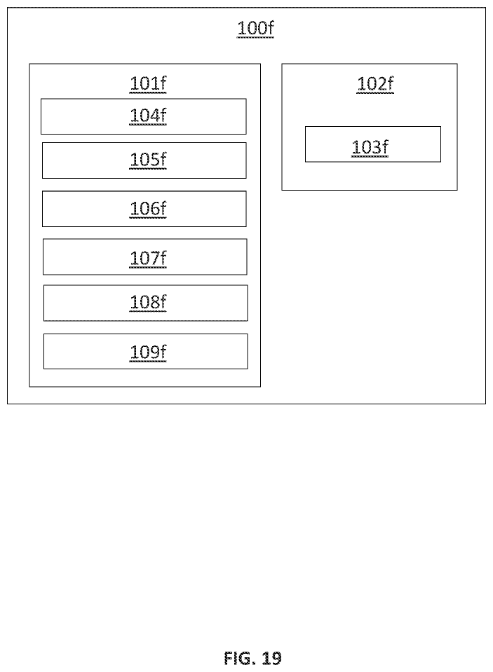

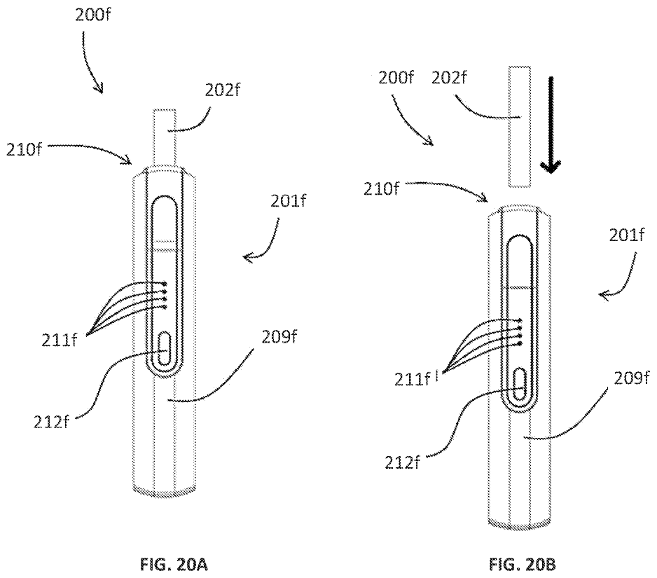

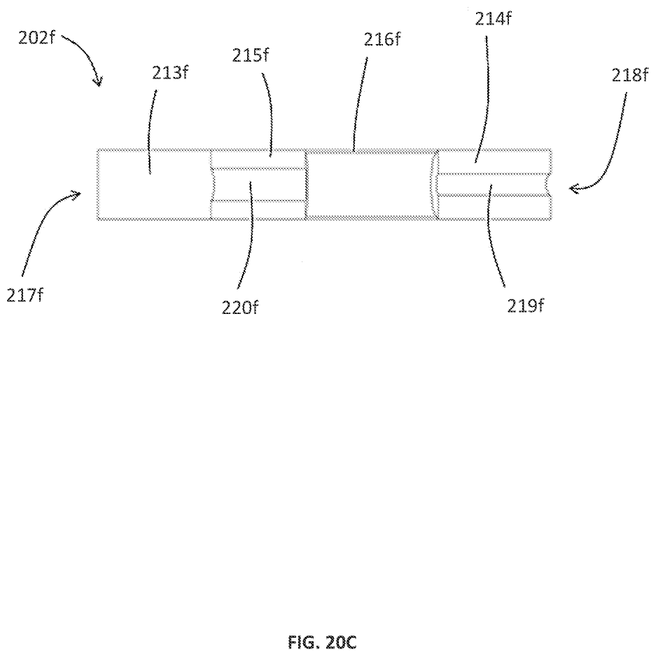

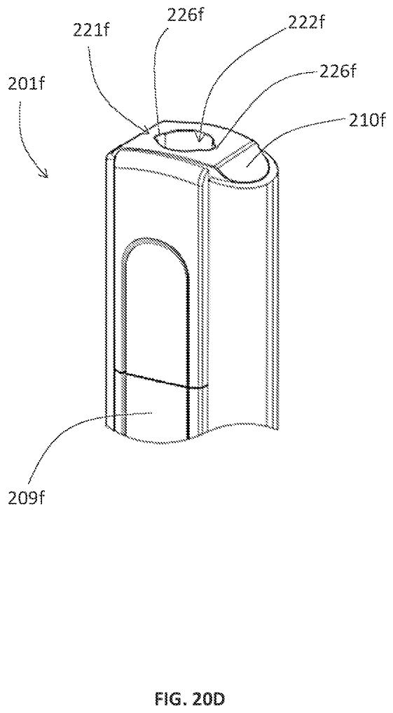

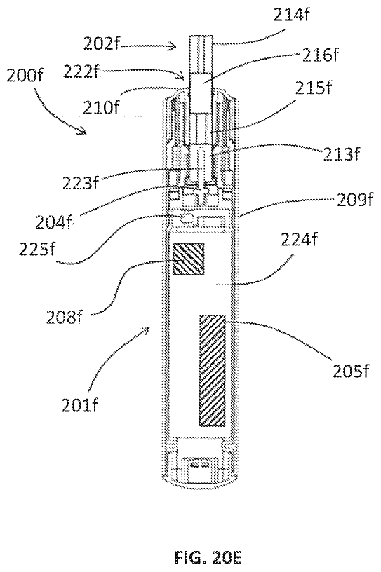

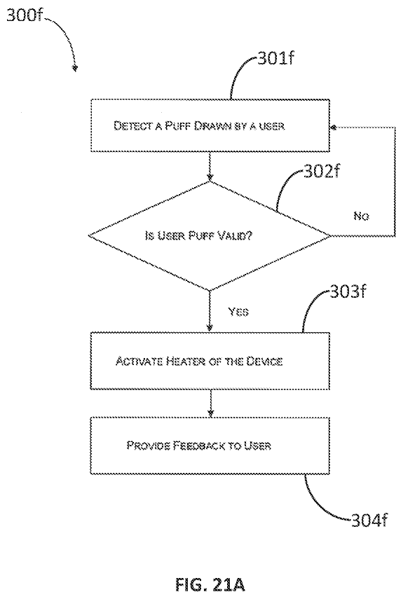

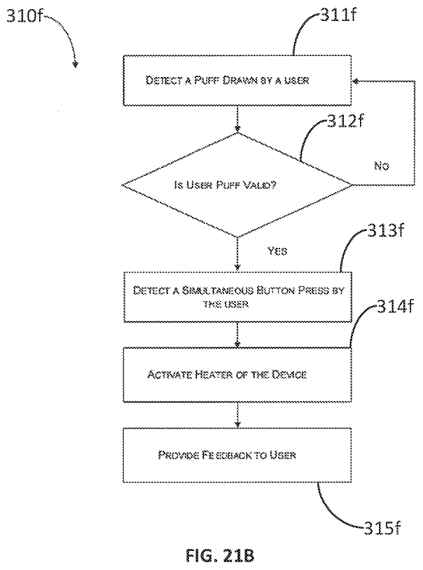

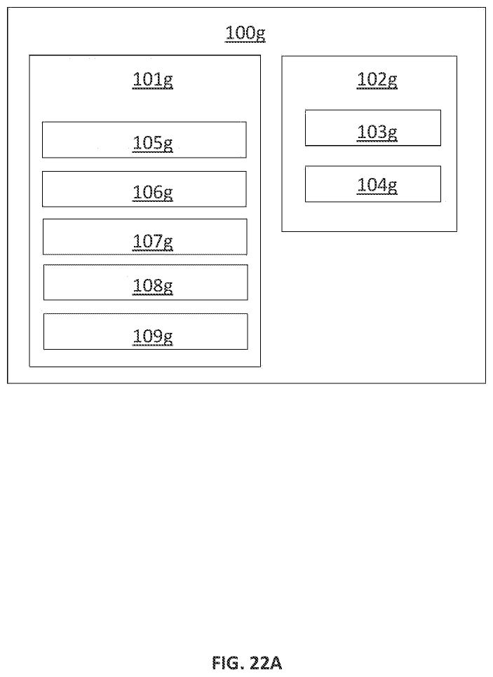

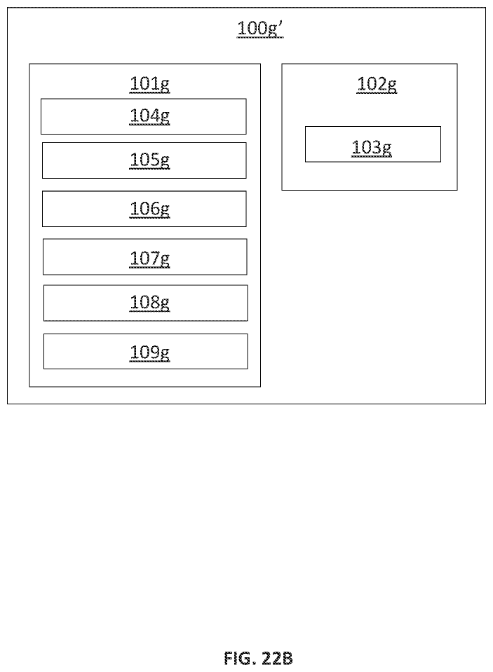

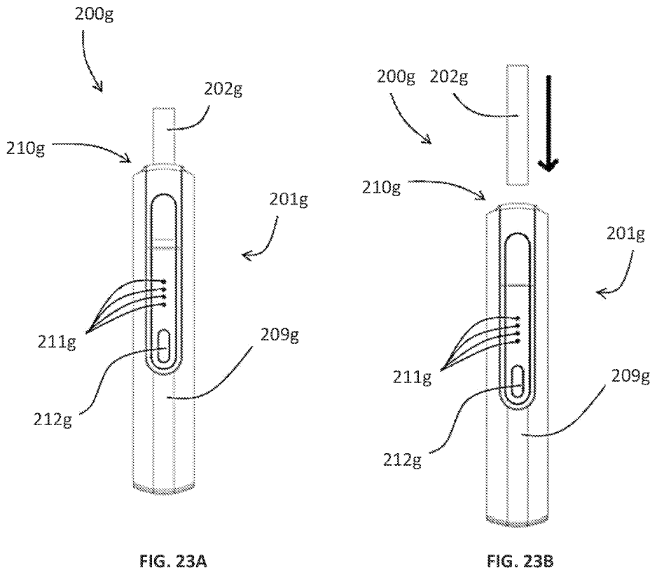

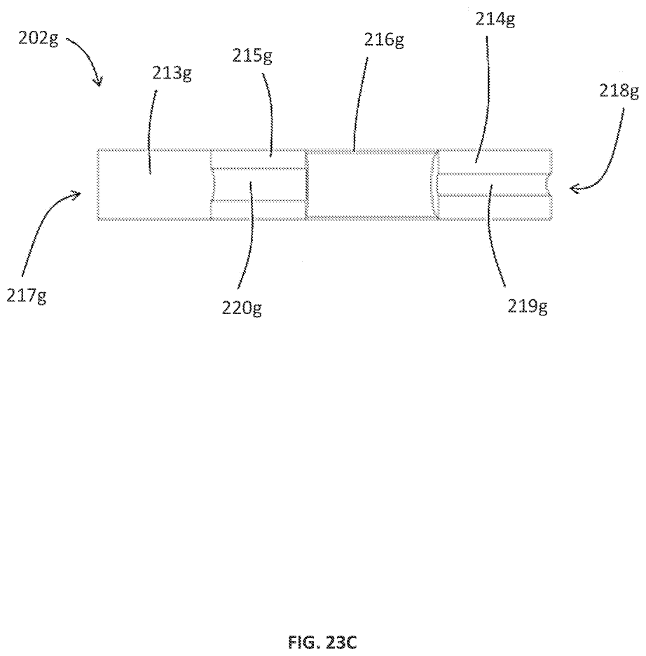

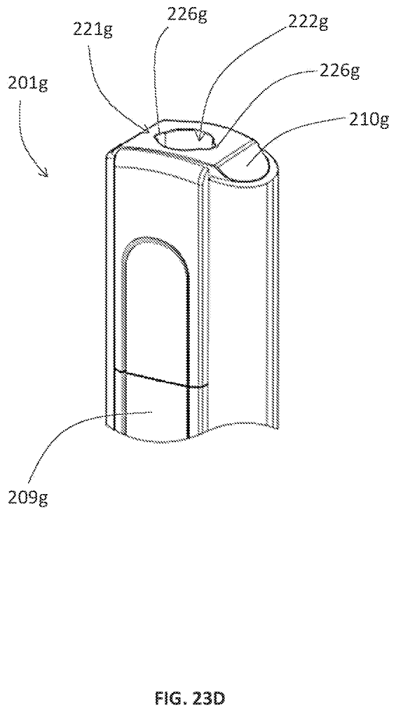

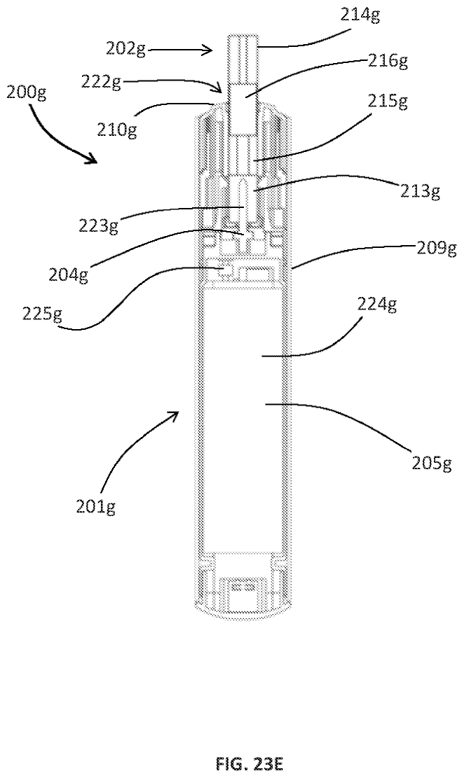

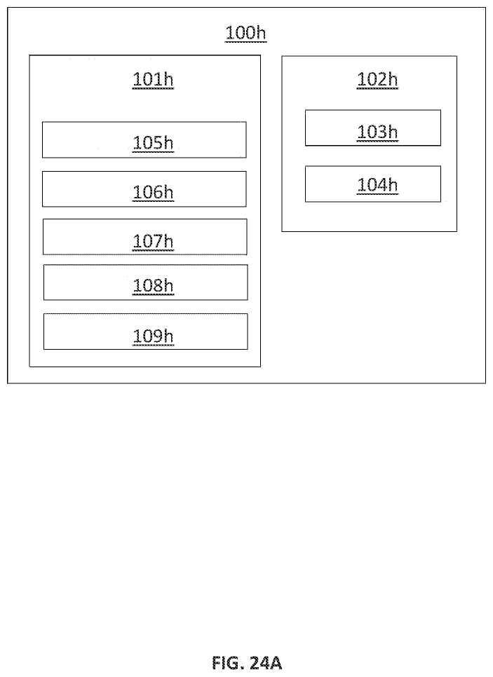

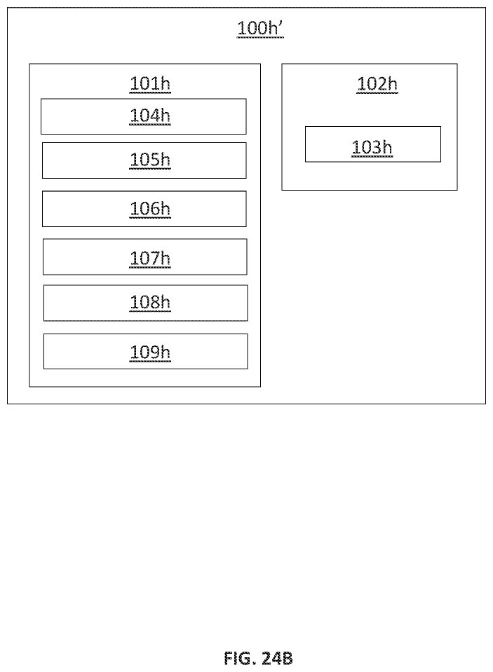

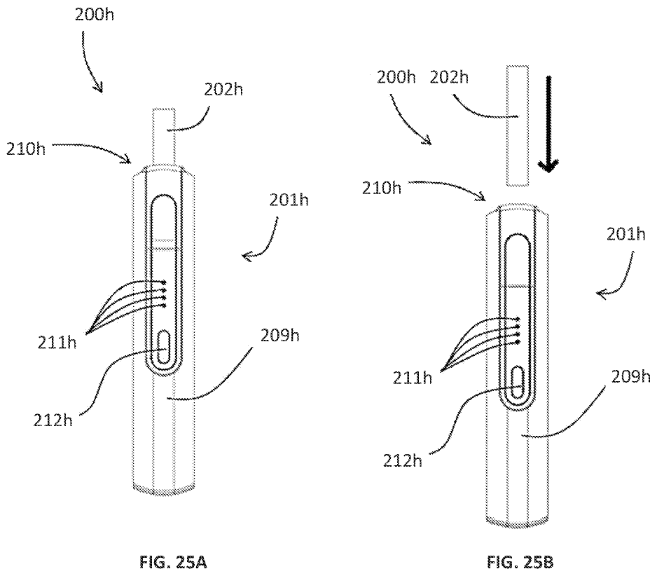

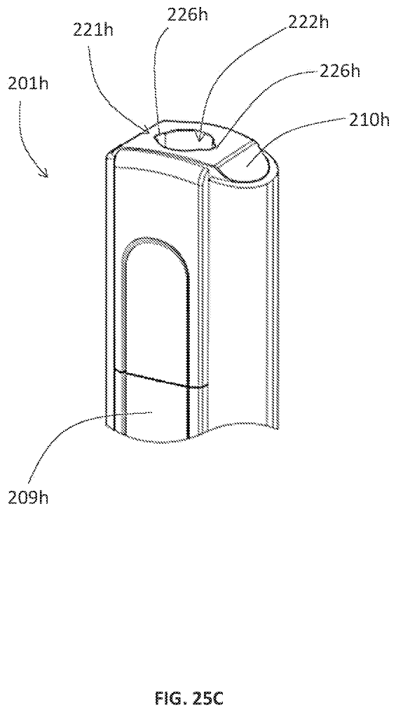

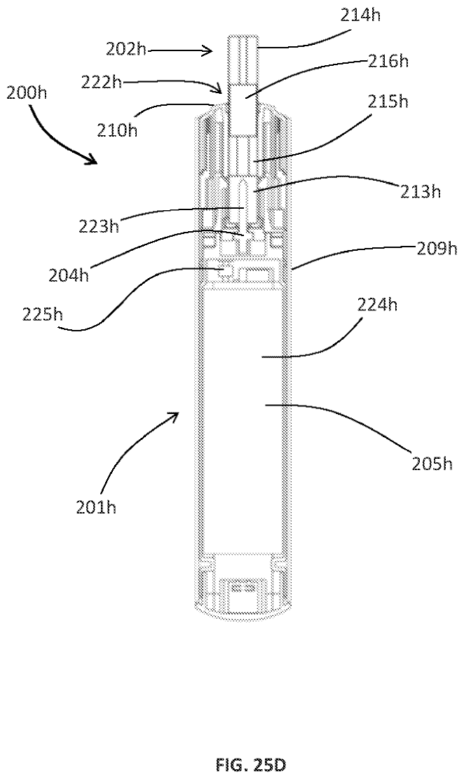

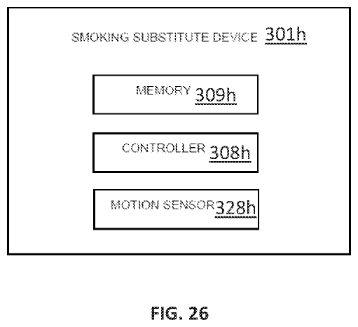

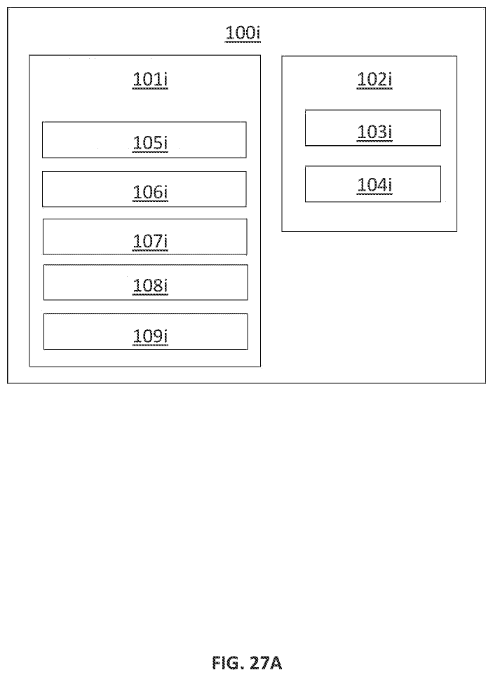

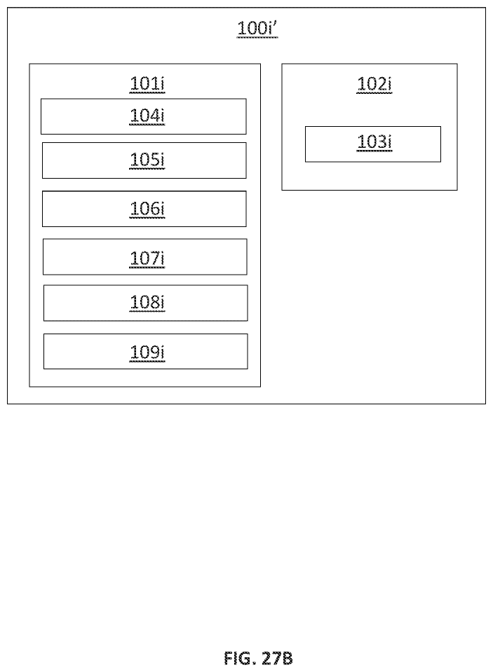

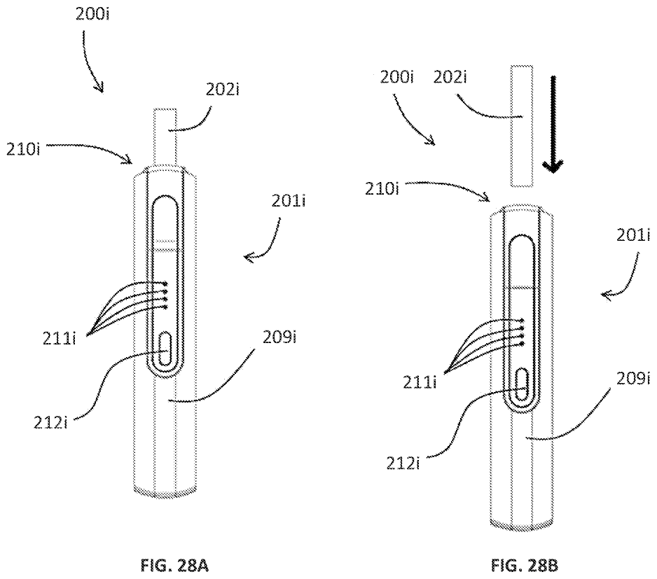

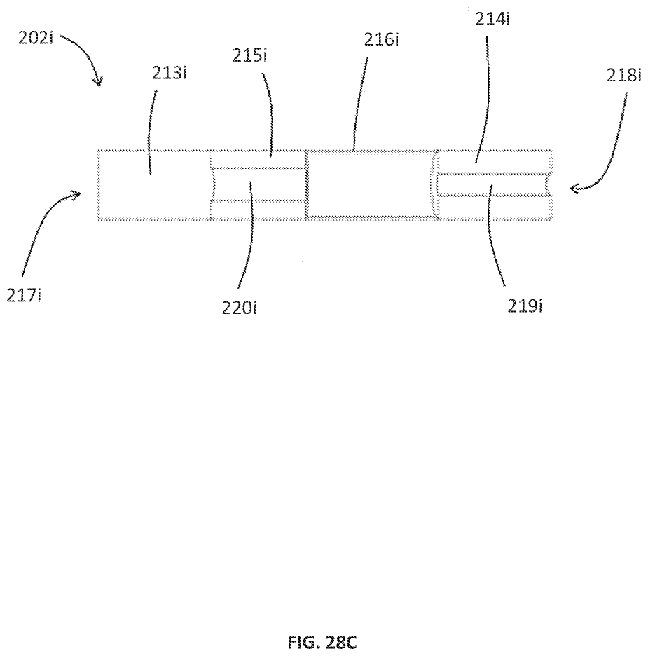

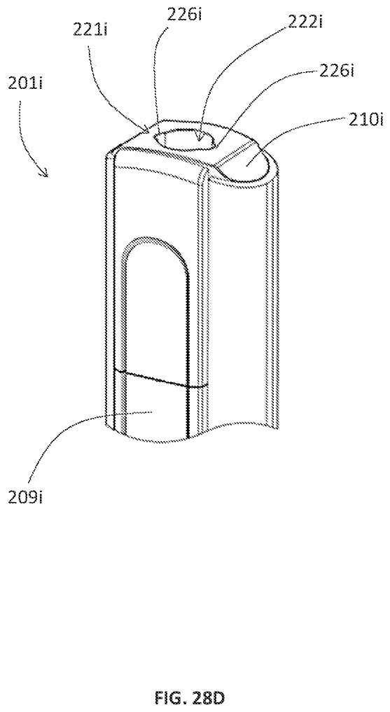

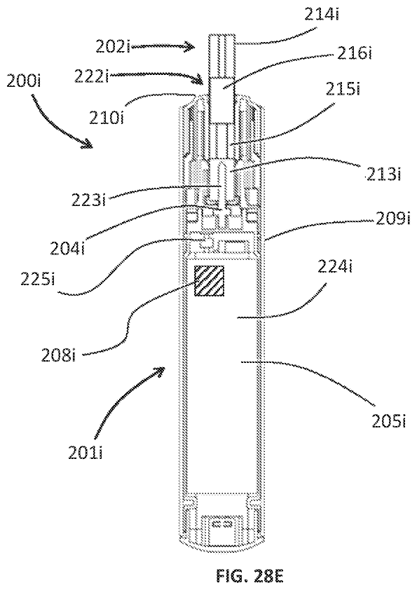

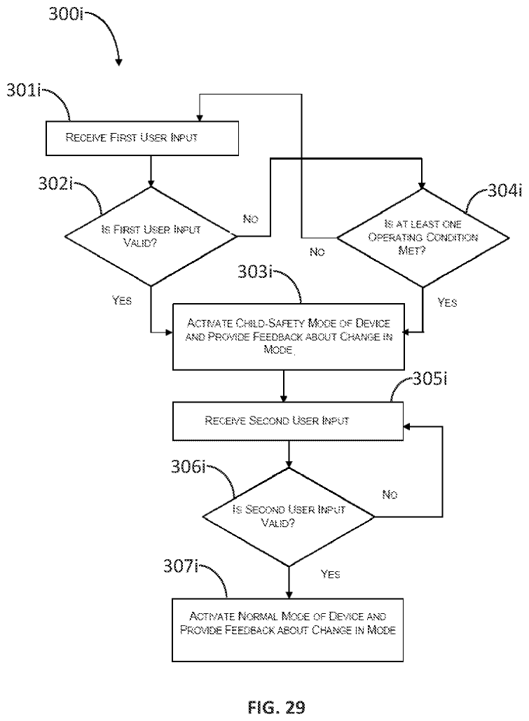

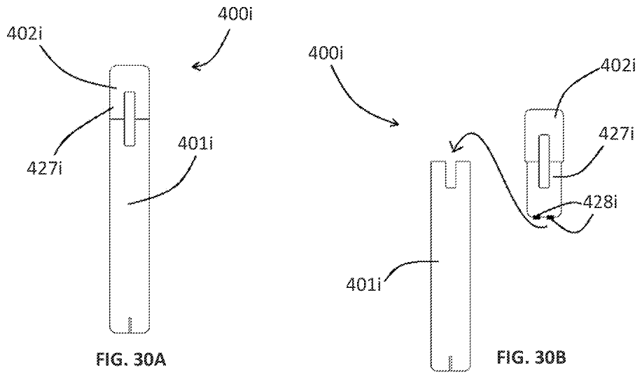

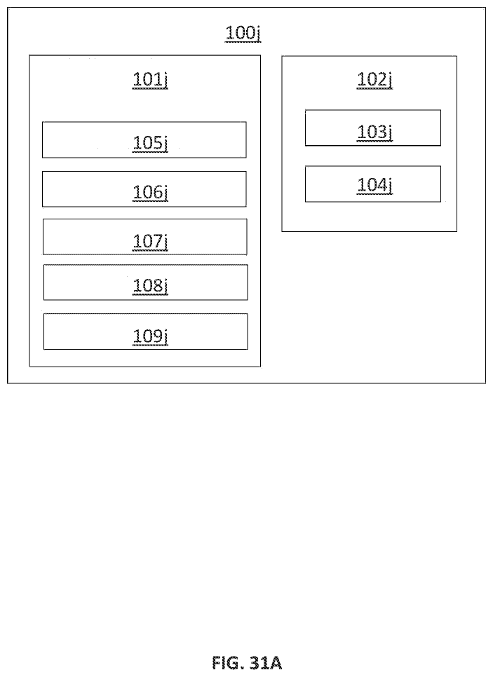

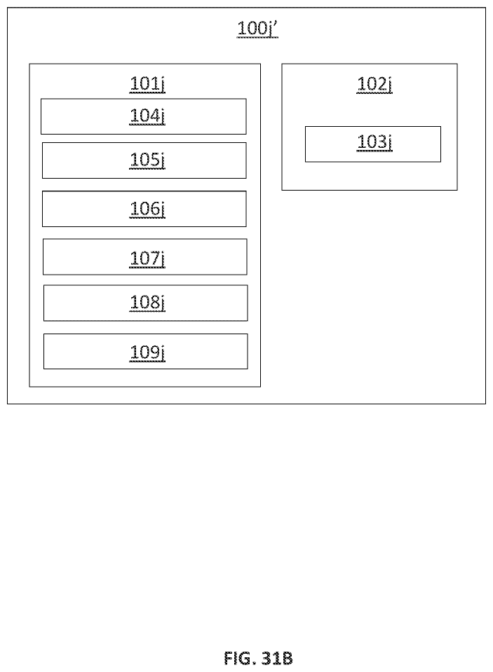

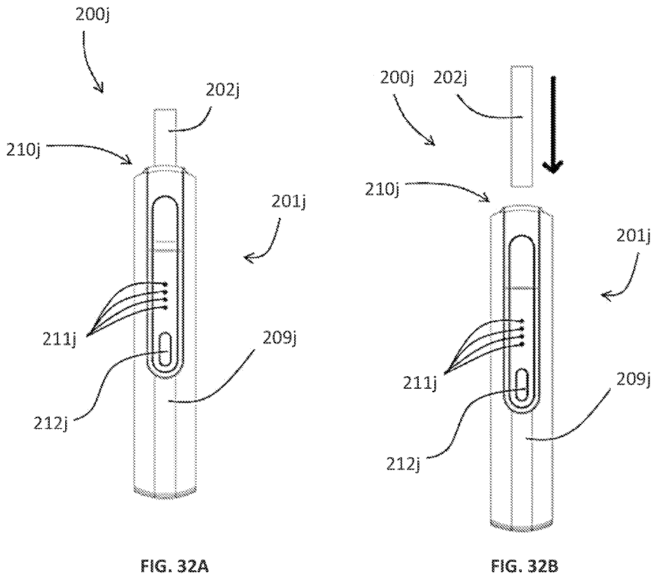

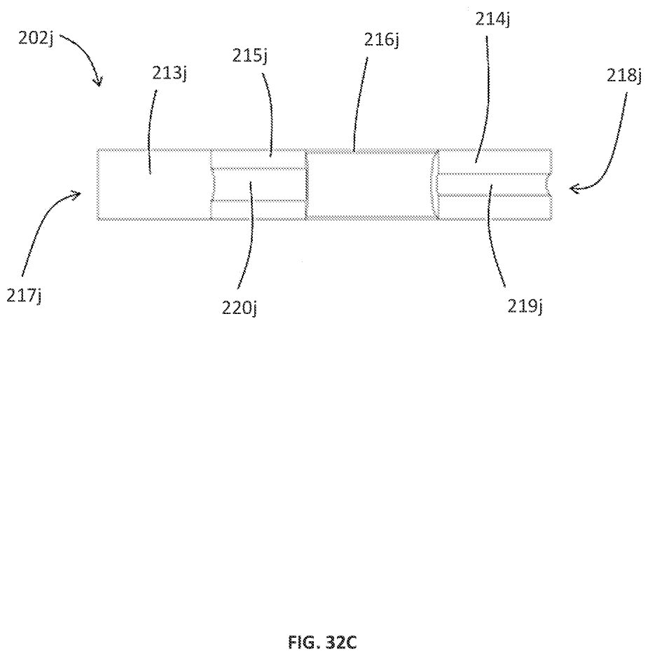

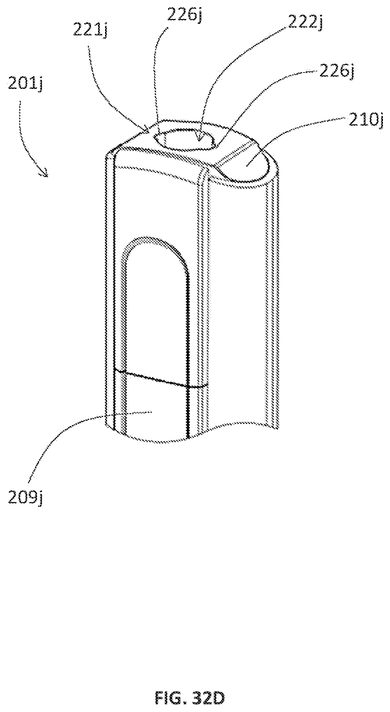

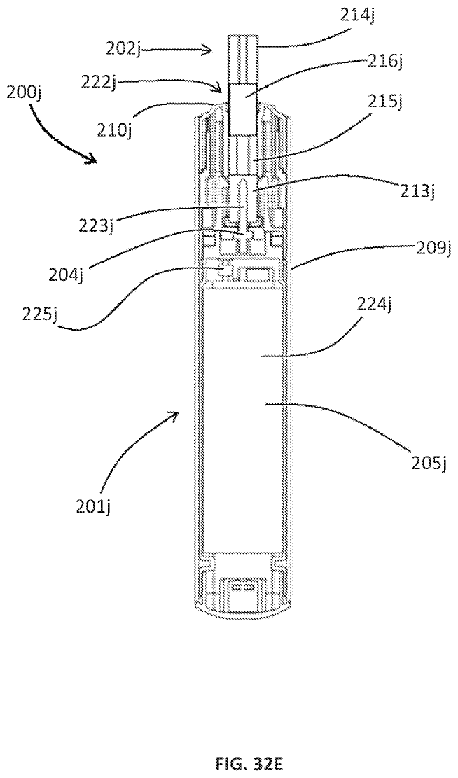

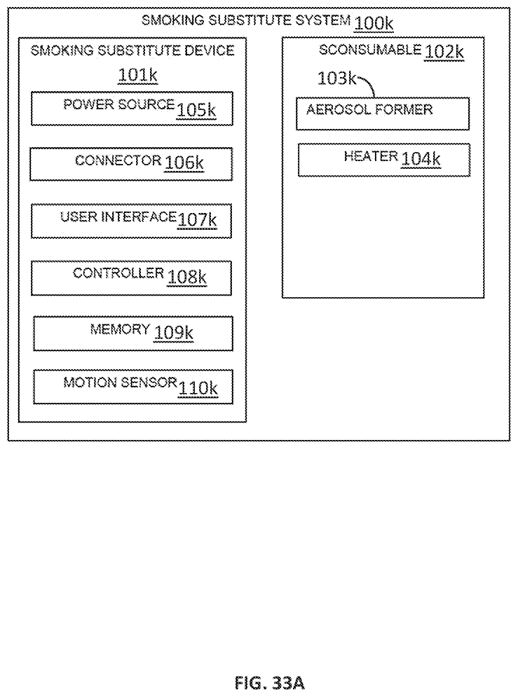

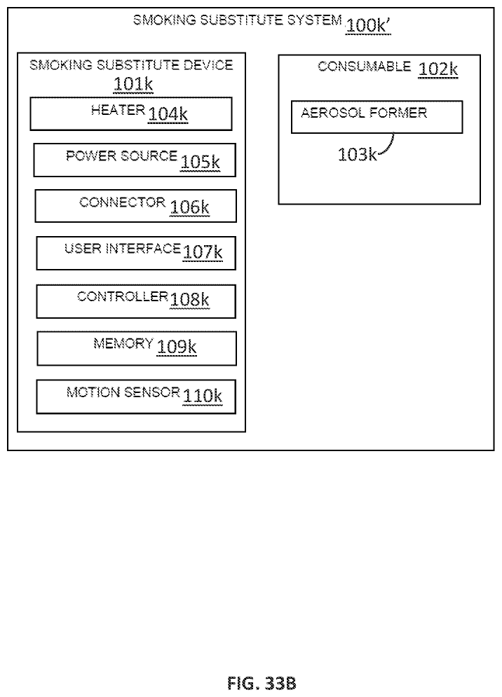

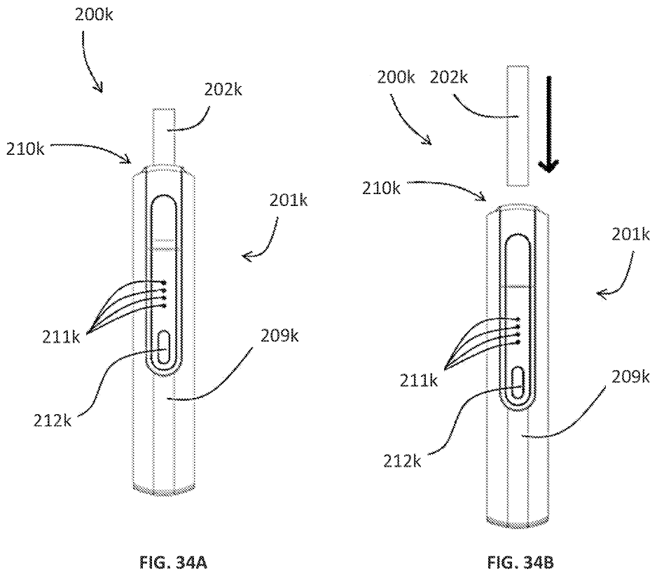

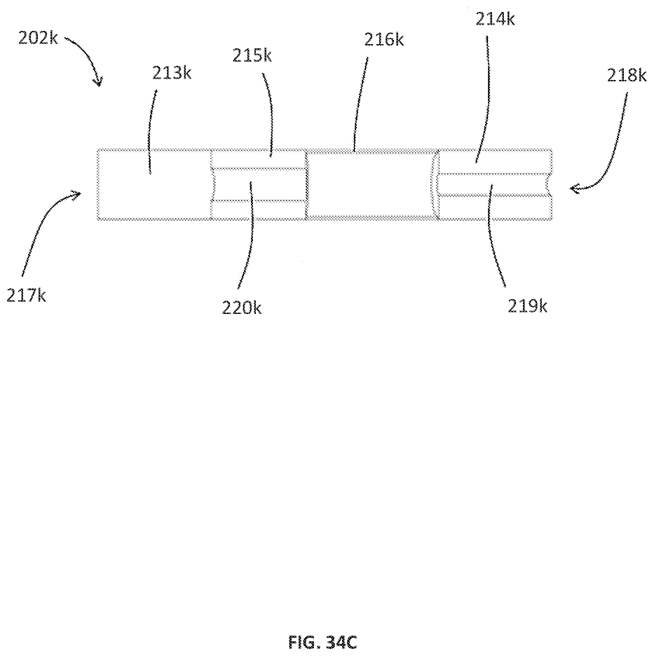

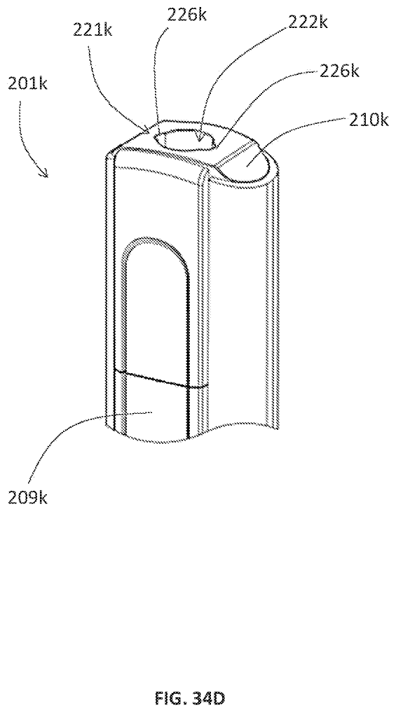

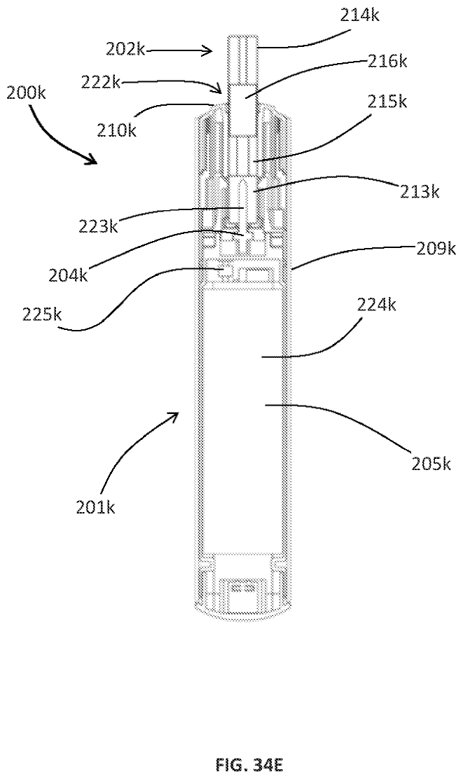

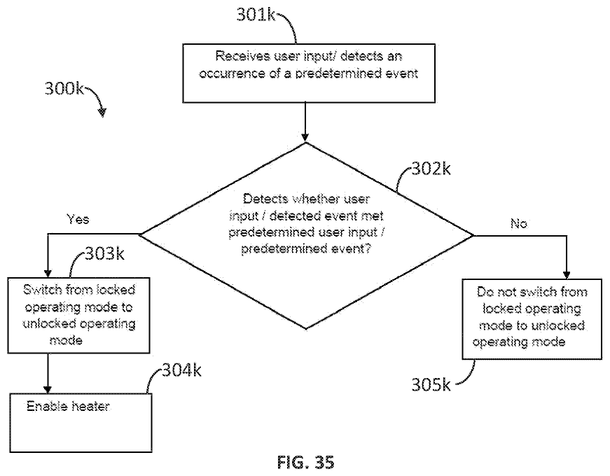

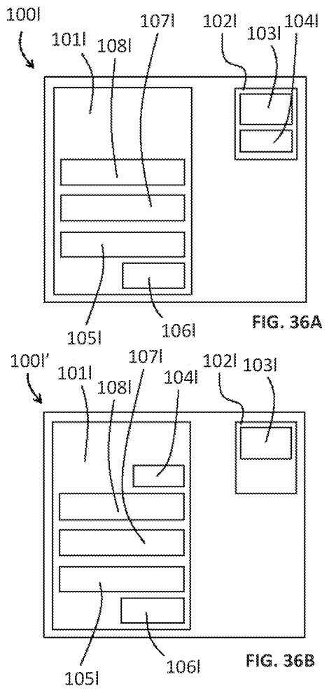

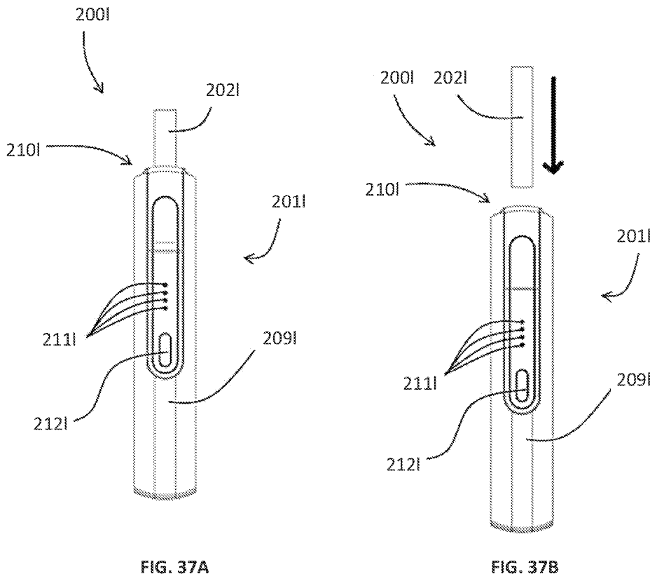

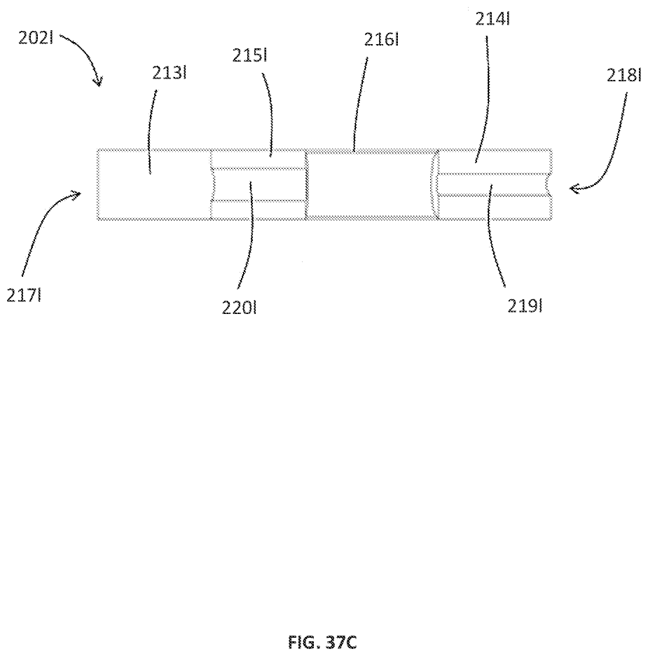

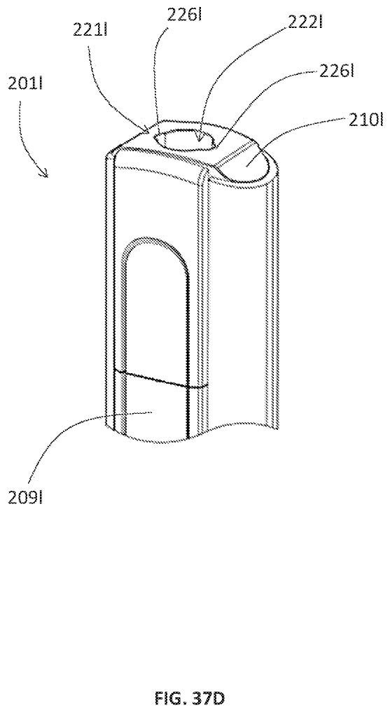

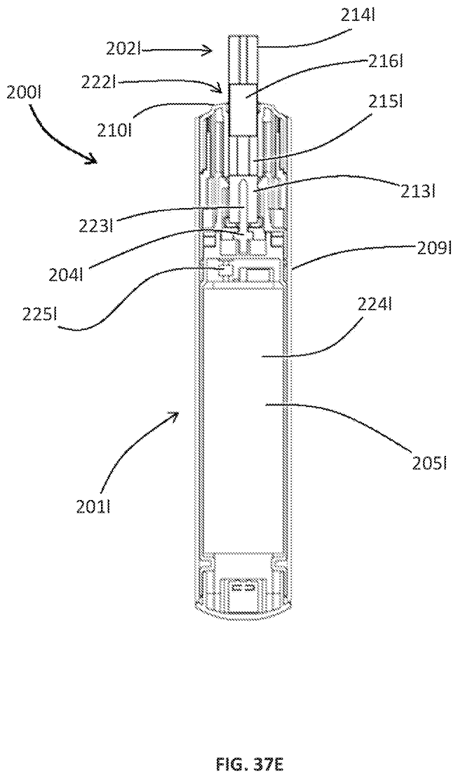

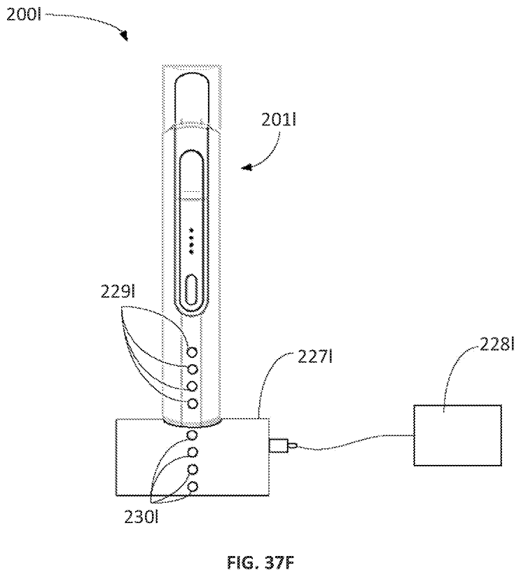

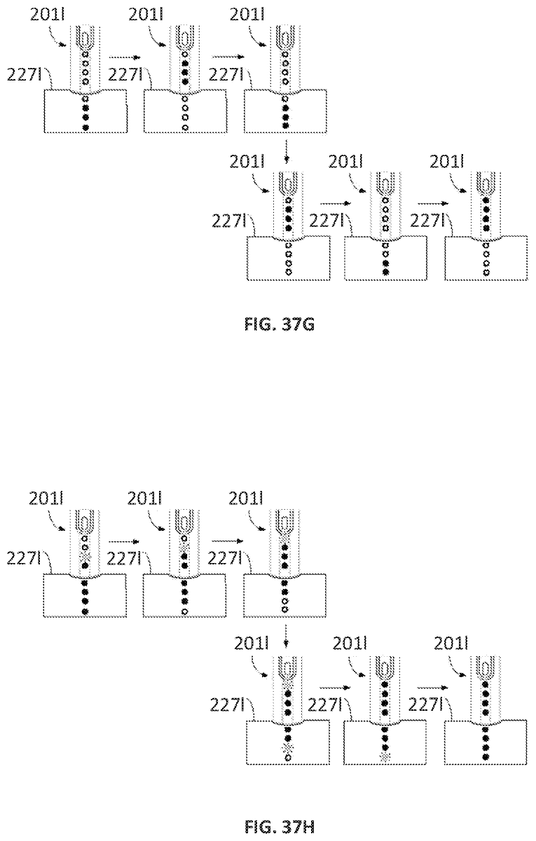

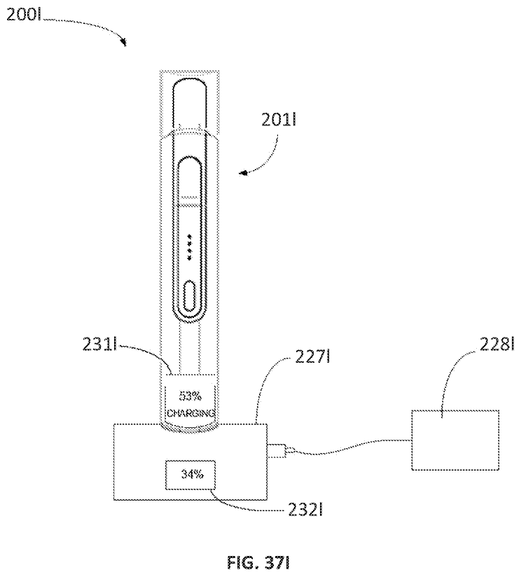

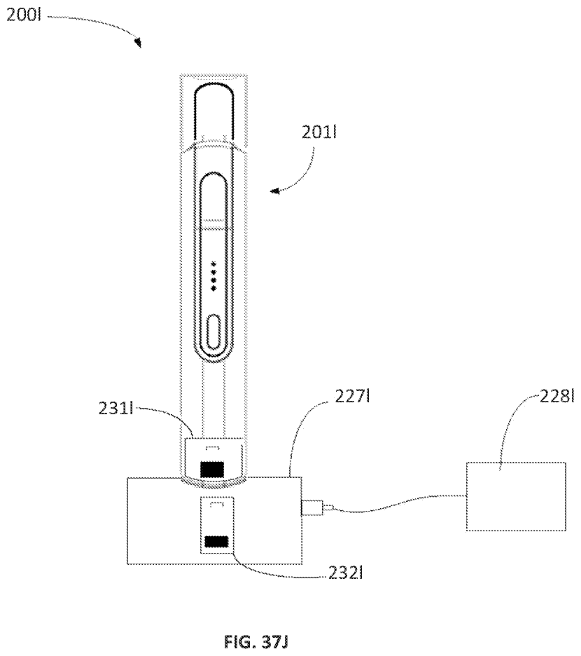

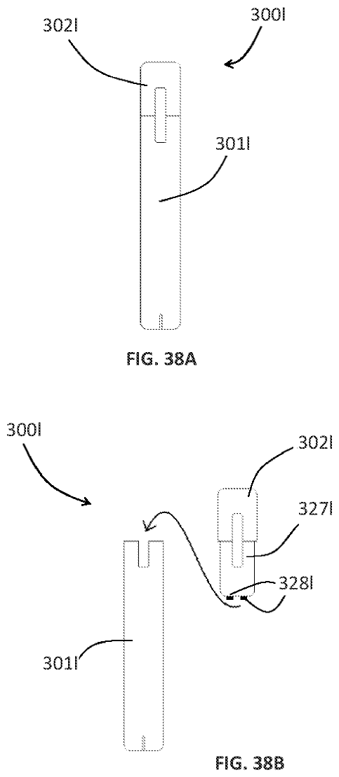

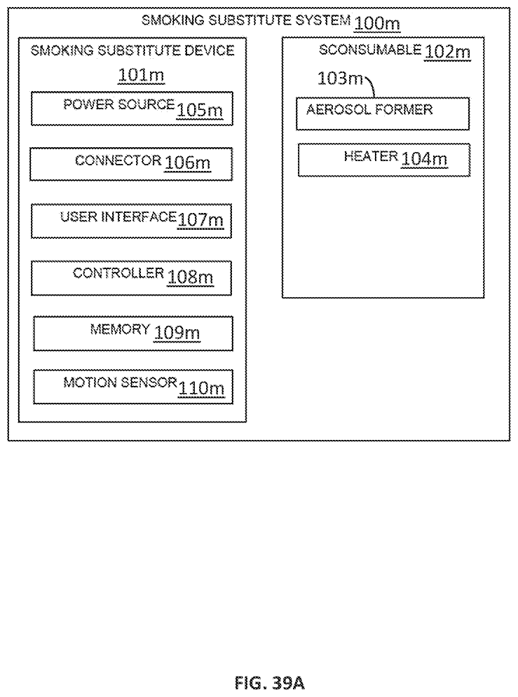

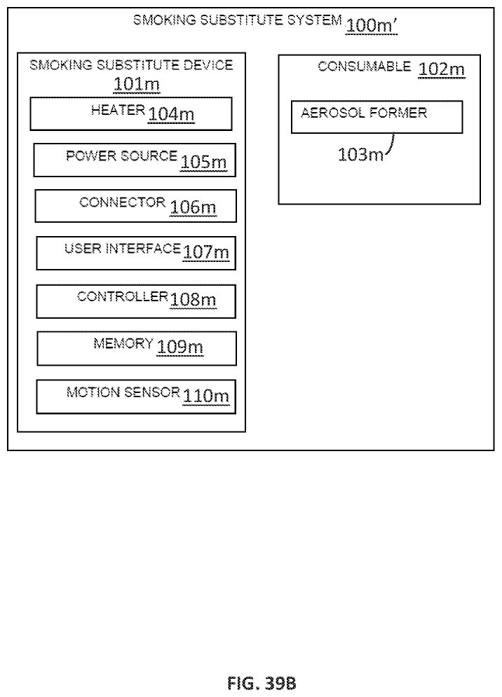

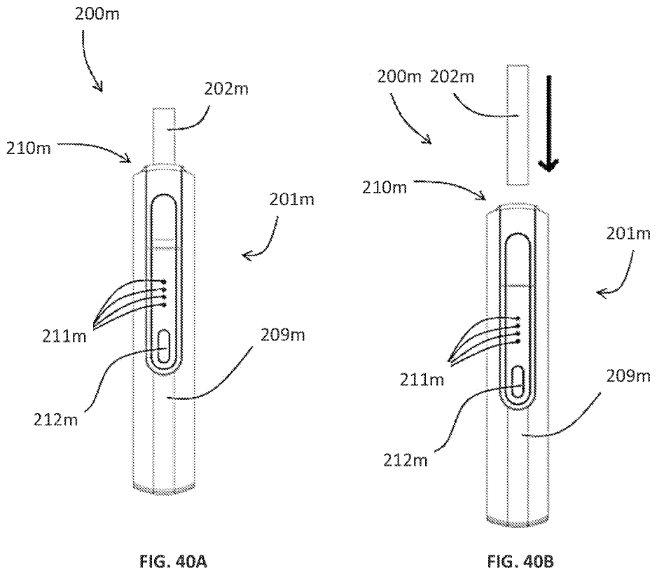

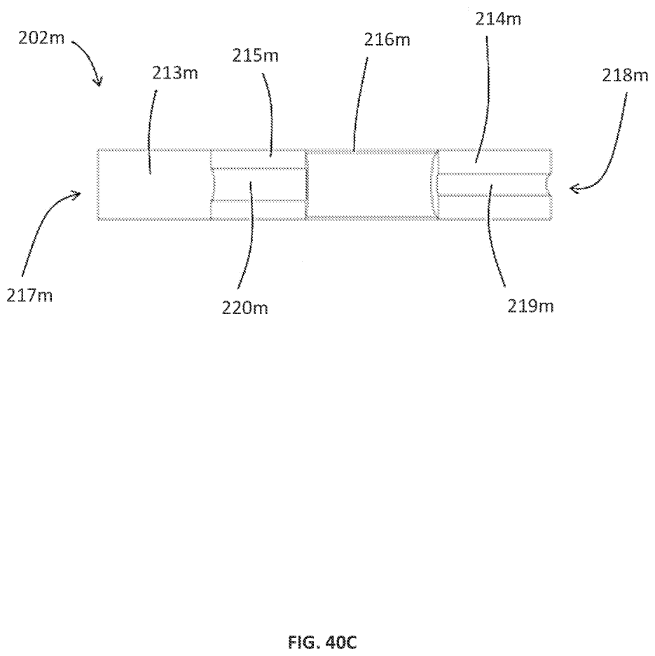

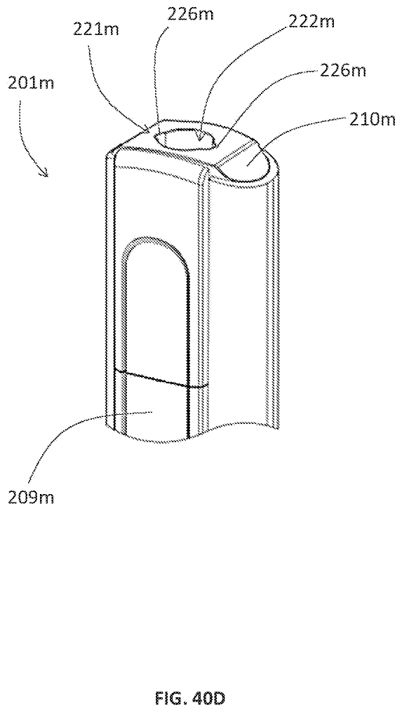

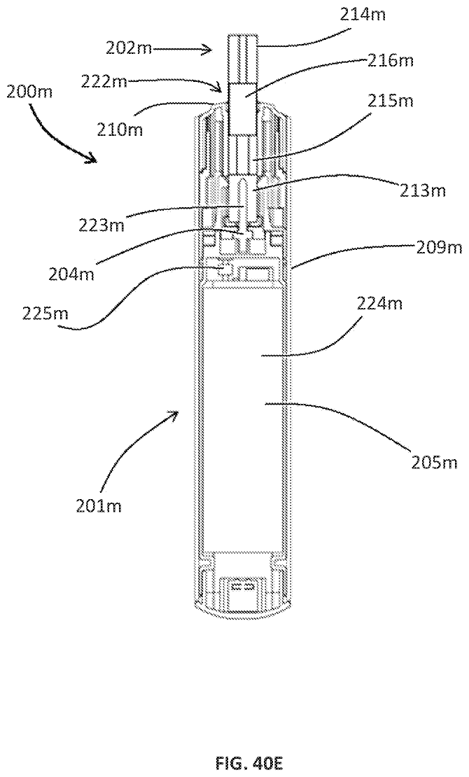

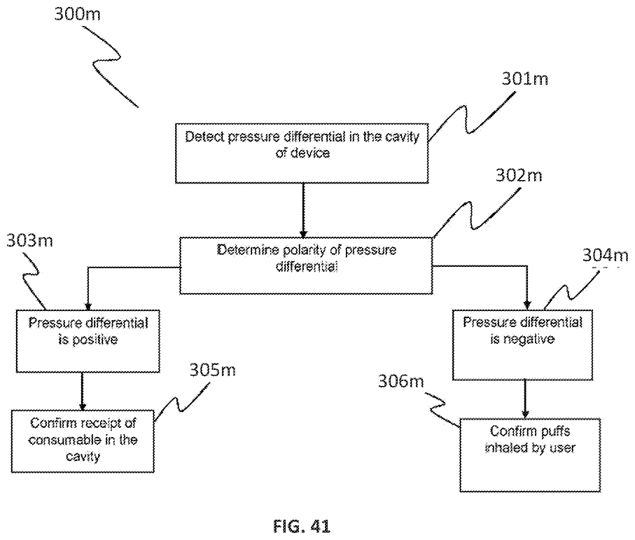

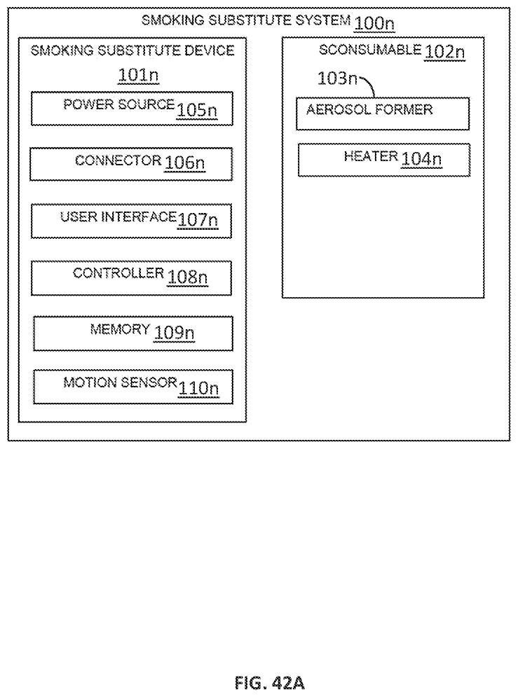

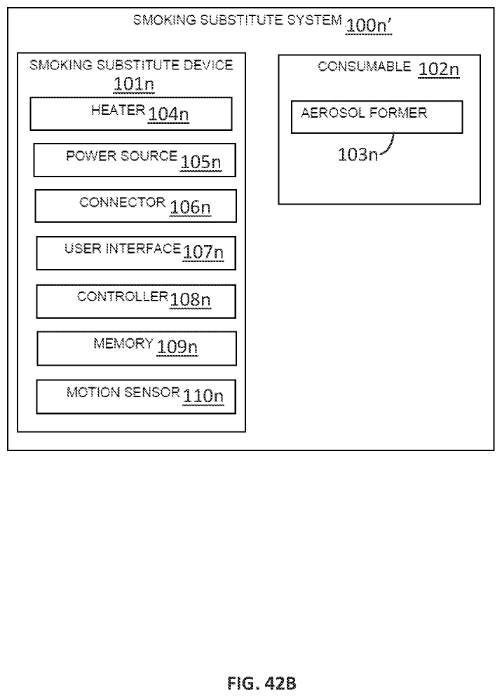

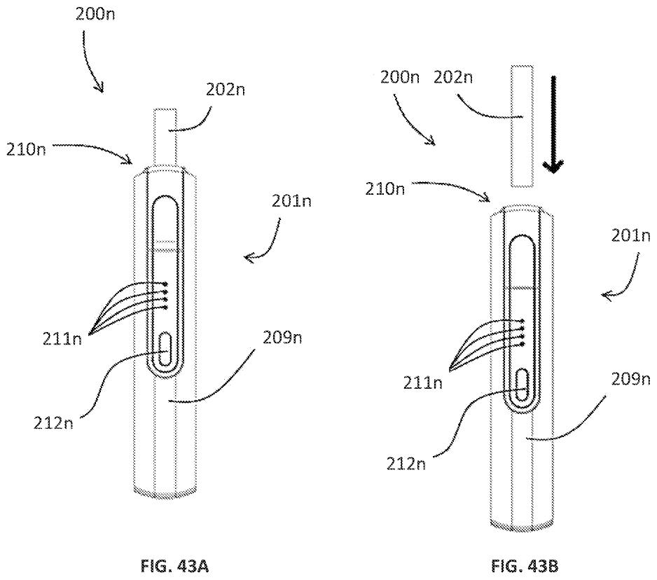

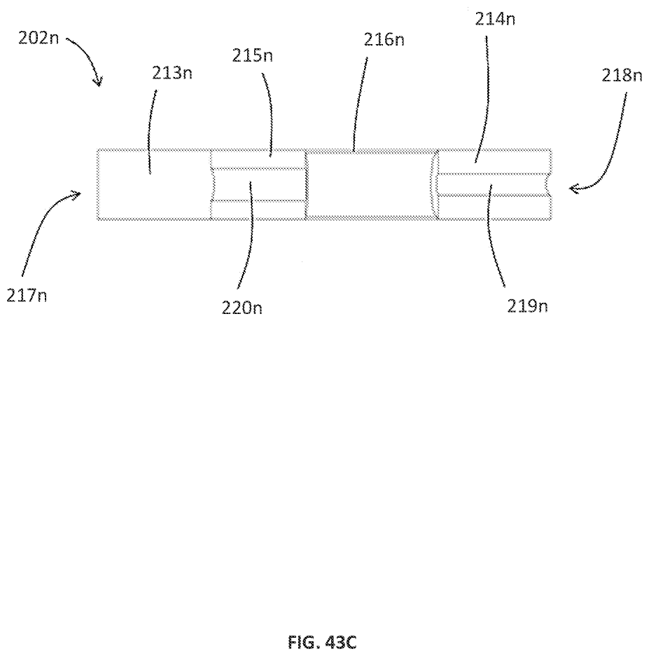

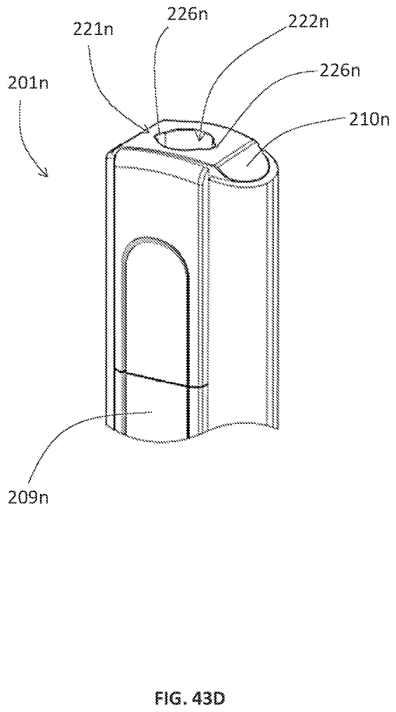

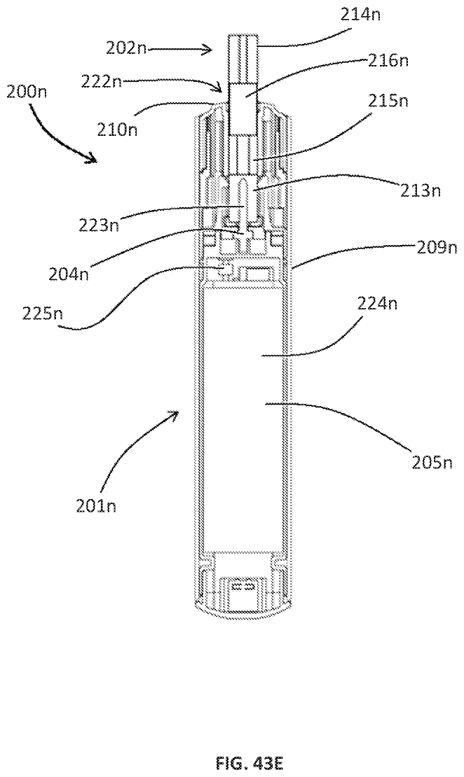

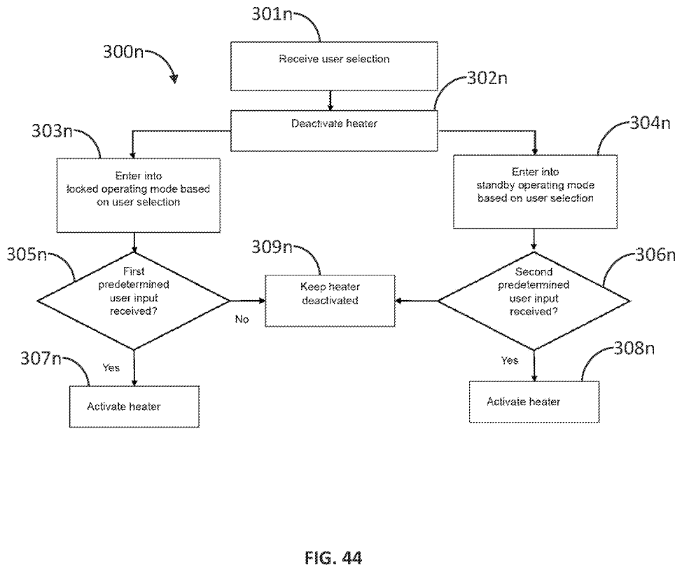

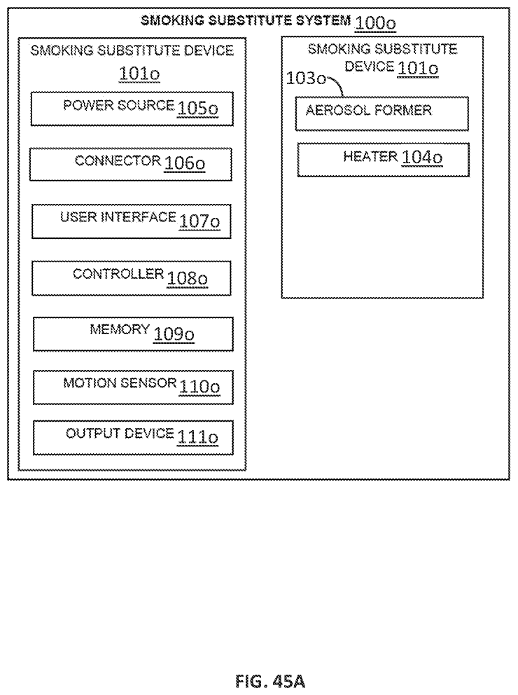

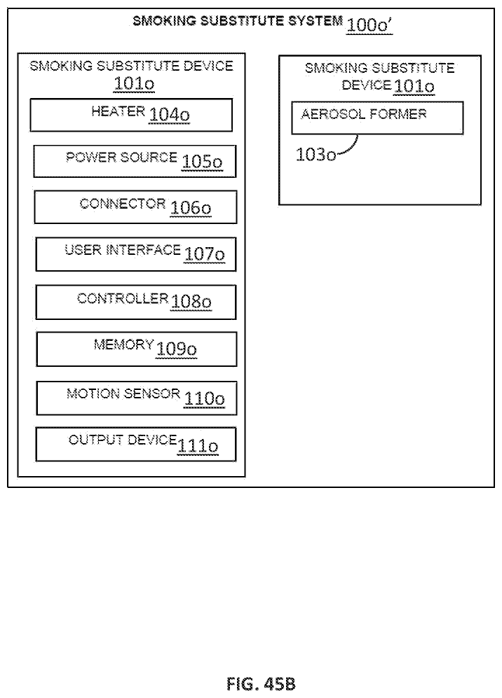

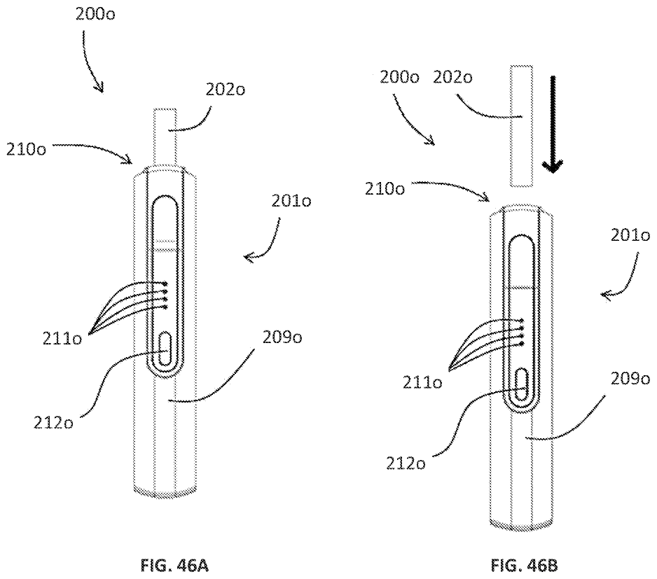

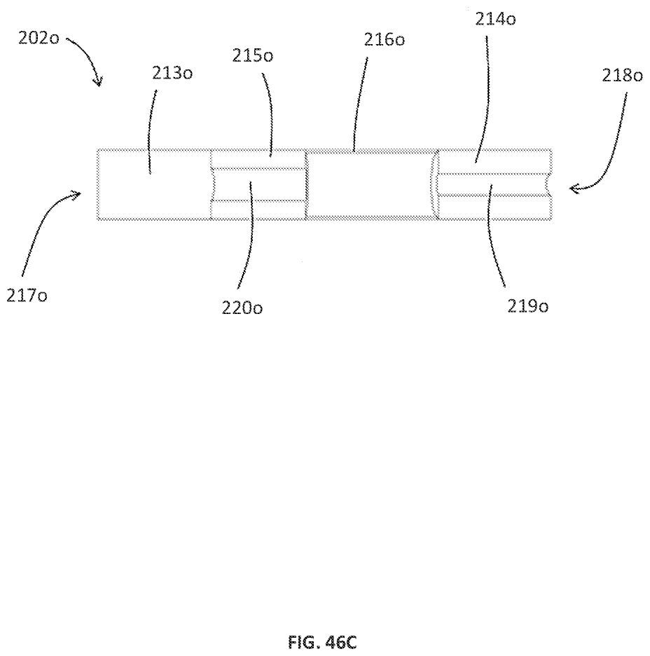

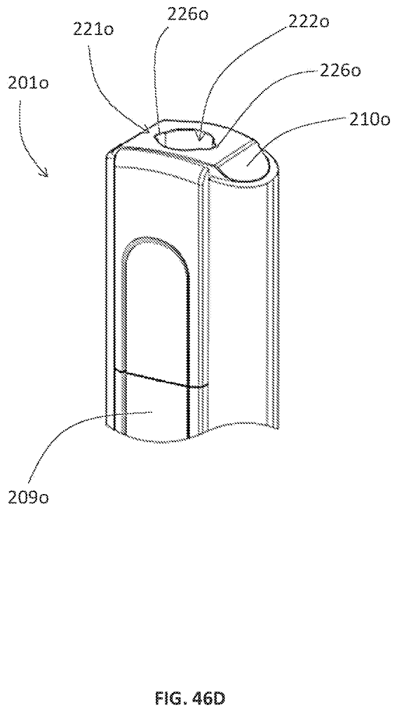

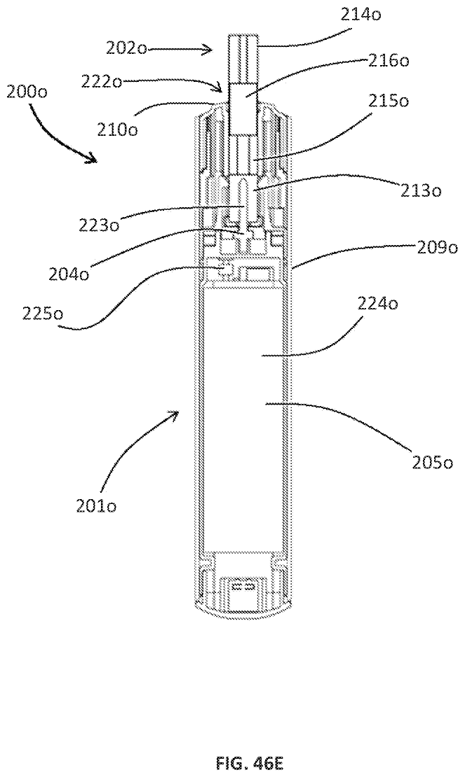

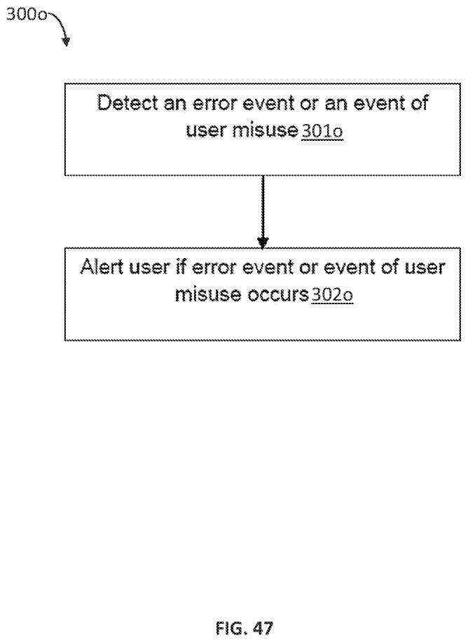

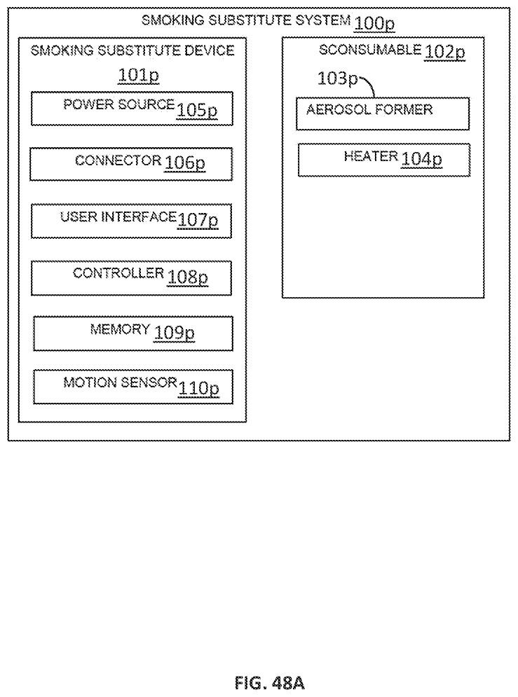

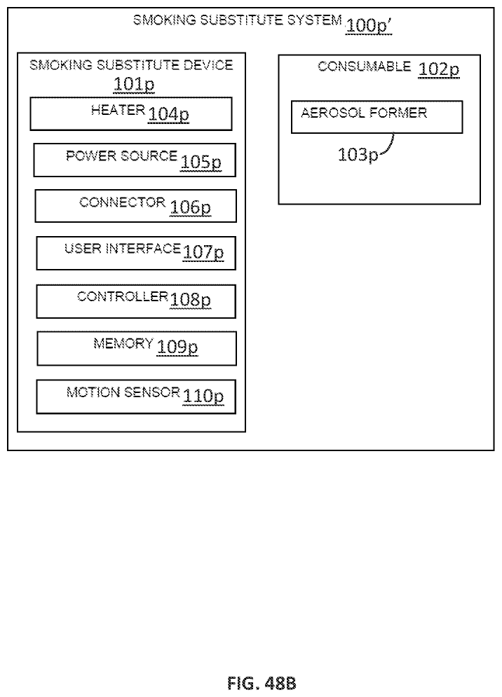

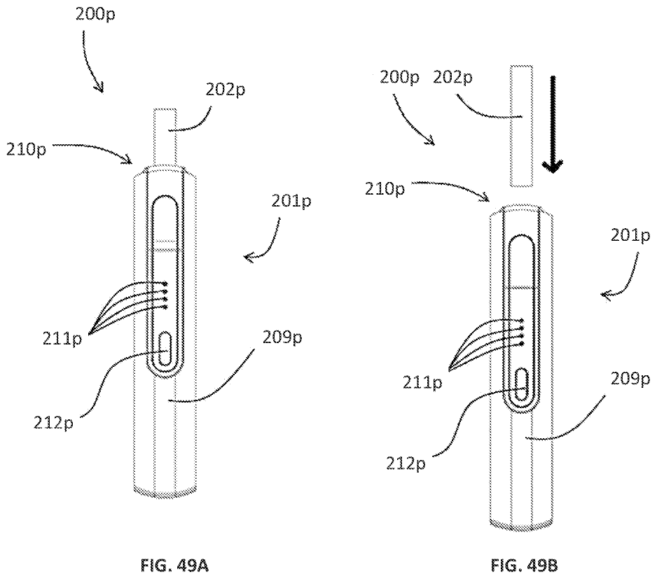

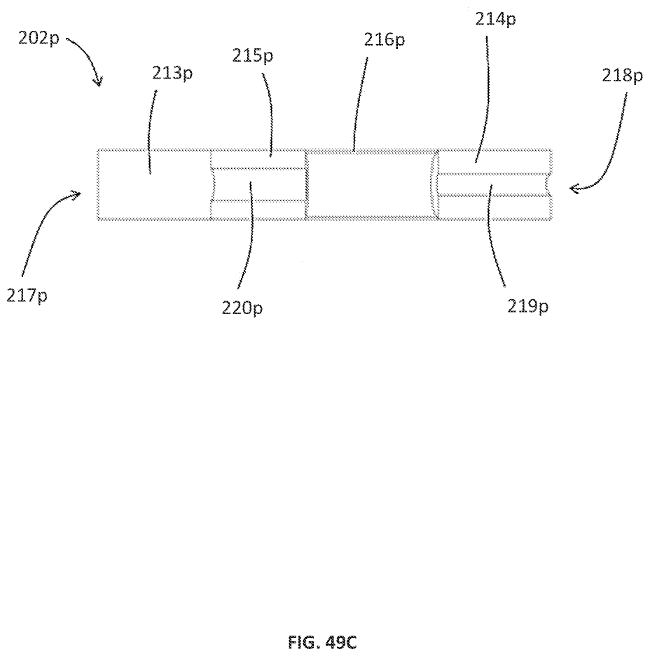

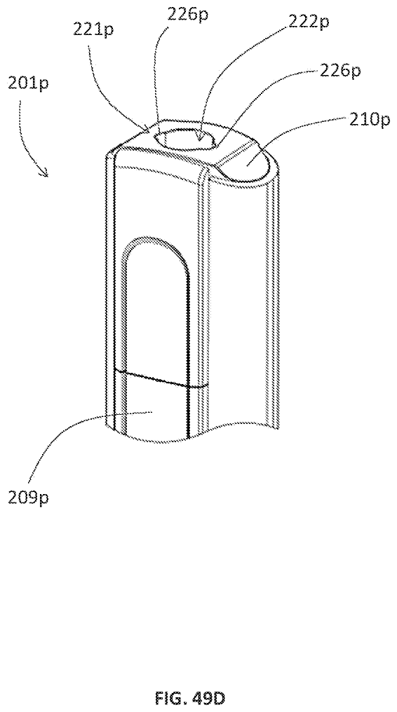

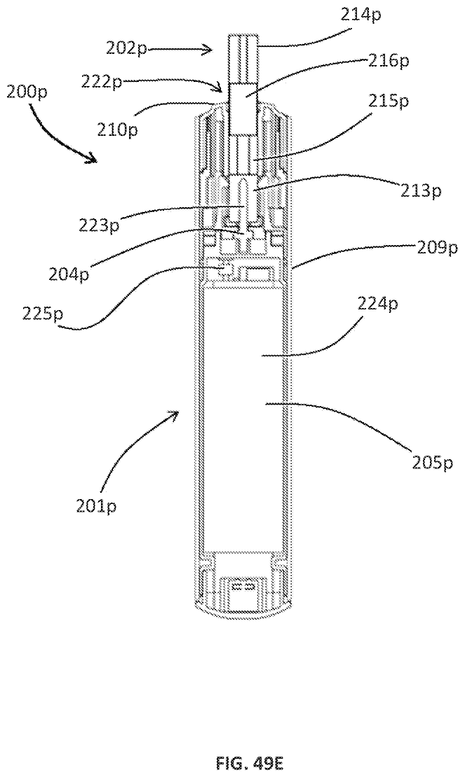

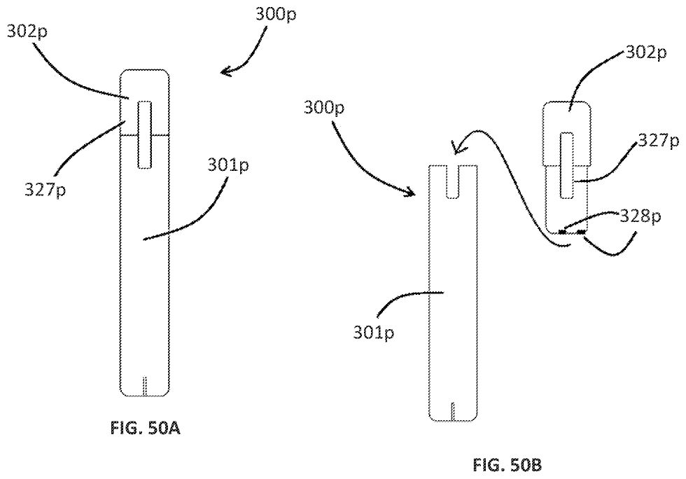

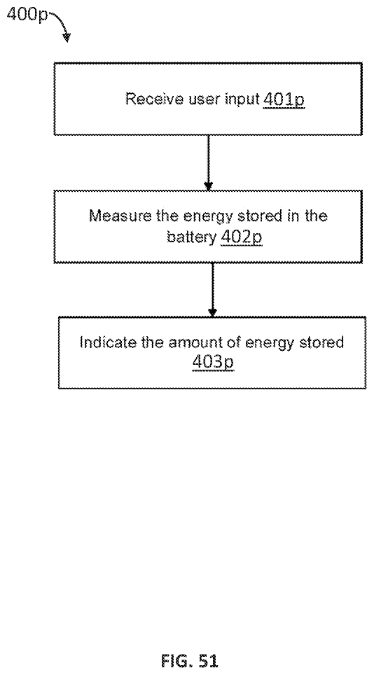

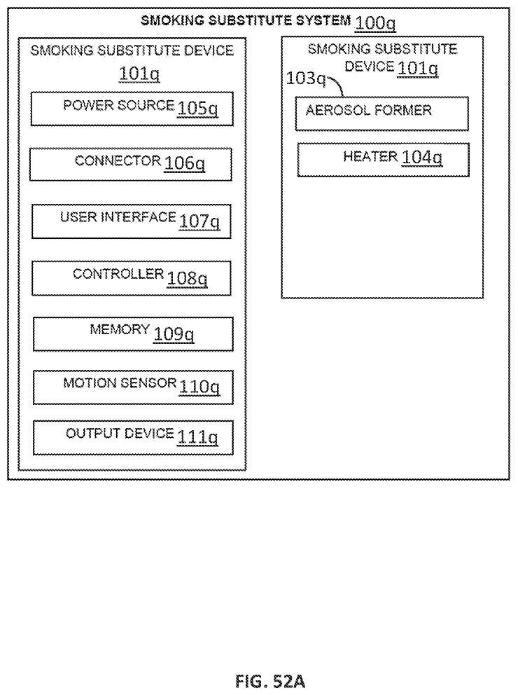

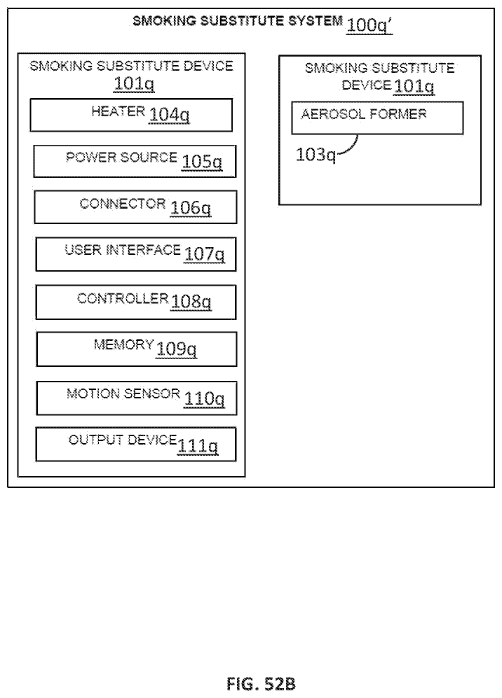

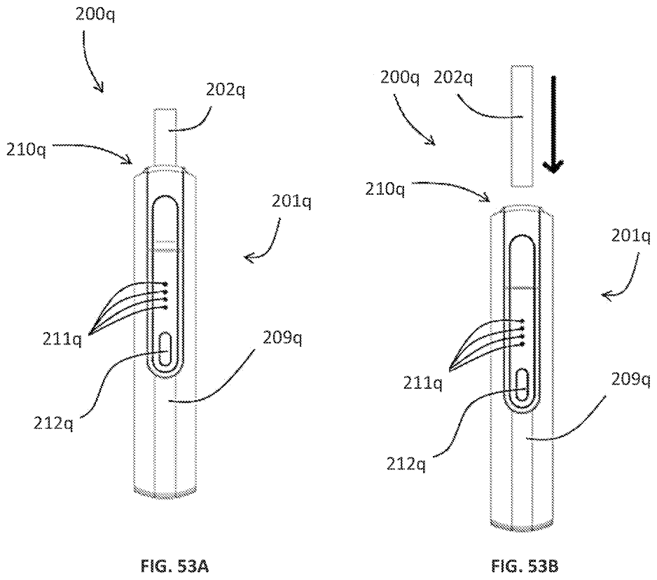

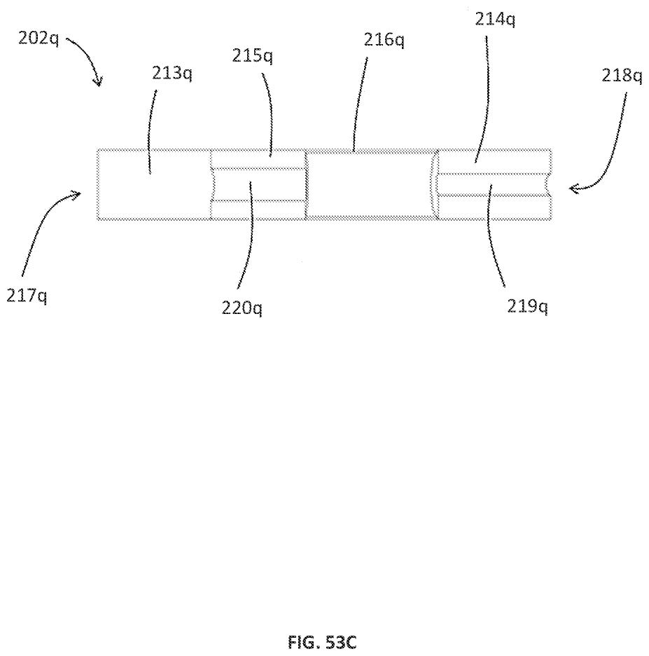

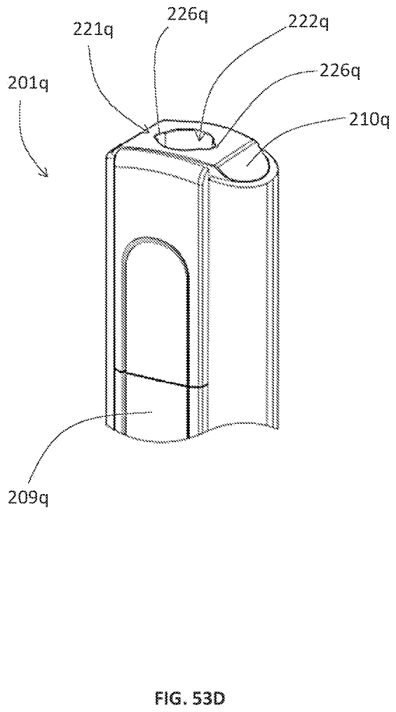

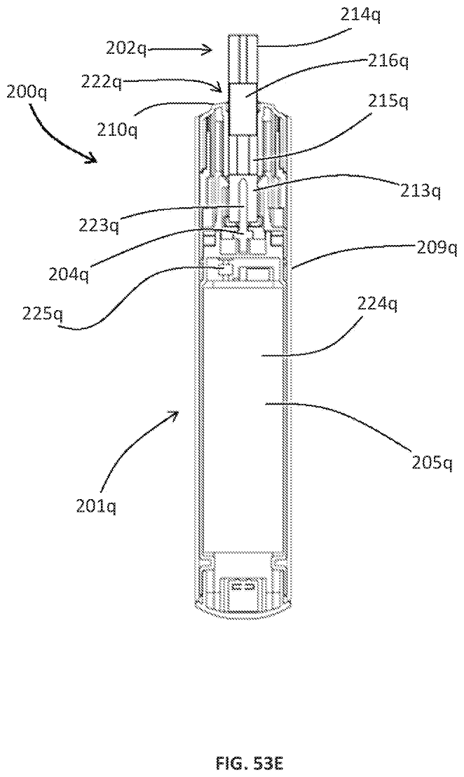

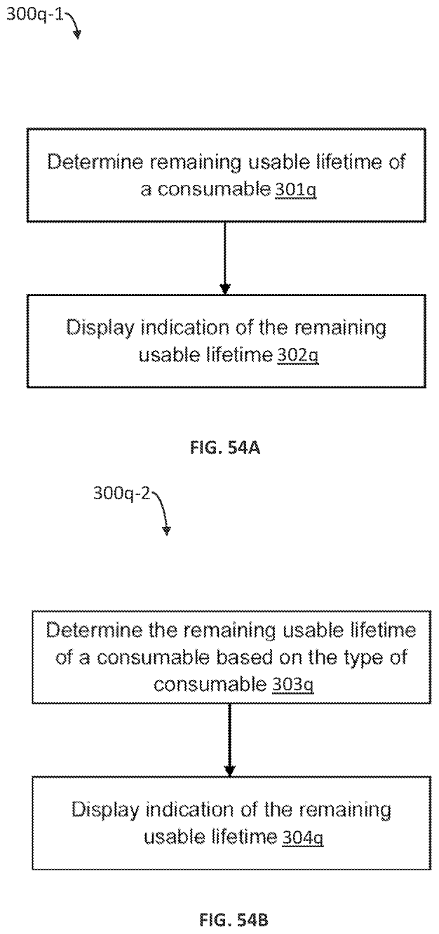

FIG. 3A and FIG. 3B illustrate flowcharts of methods 300-1 and 300-2 of determining the remaining usable lifetime of a consumable.

As illustrated in FIG. 3A and FIG. 3B, the methods 300-1 and 300-2 each include one or more blocks implemented by the controller 208 of the device 201. The methods may be described in general context of controller executable instructions. Generally, controller executable instructions may include routines, programs, objects, components, data structures, procedures, modules, and functions, which perform particular functions or implement particular abstract data types.

The order in which the methods 300-1 and 300-2 are described is not intended to be construed as a limitation, and any number of the described method blocks can be combined in any order to implement the methods. Additionally, individual blocks may be deleted from the methods without departing from the scope of the subject matter described herein. Furthermore, the methods can be implemented in any suitable hardware, software, firmware, or combination thereof.

Referring to FIG. 3A, at block 301, the controller 208 is configured for determining the remaining usable lifetime of a consumable during a smoking session. Although not explicitly disclosed in the flowchart, for determining the remaining usable lifetime of a consumable 202 in said session, the controller 208 may be configured to incorporate one or more of (i) known (e.g., average) usable lifetimes of consumables, (ii) remaining duration of the consumable cycle, (ii) ambient temperature, (iii) number and/or magnitude of puff inhalations made by the user, and (iv) type of consumable present in the device.

At block 302, the controller 208 is configured for displaying, to the user, an indication of the remaining usable lifetime of the consumable 202, as determined by the controller 208 at block 301. In some embodiments, the controller 208 may be configured for determining and displaying the remaining usable lifetime of the consumable 202 in response to receiving user input/request.

Referring to FIG. 3B, at block 303, the controller 208 is configured for determining the remaining usable lifetime of a consumable 202 during a smoking session based on the type of consumable. Although not explicitly disclosed in the flowchart, however, before determining the remaining usable lifetime based on the type of consumable, the controller 208 is configured for determining the type of consumable inserted into the device 201. In some embodiments, the type of consumable may be determined by one or more different types of sensors placed inside the device 201.

In some embodiments, for determining the remaining usable lifetime of a consumable 202, the controller 208 may be configured to additionally use one or more of (i) known (e.g., average) usable lifetimes of consumables of the type detected, (ii) remaining duration of the consumable cycle, (iii) ambient temperature and (iv) number and/or magnitude of puff inhalations made by the user.

At block 304, the controller 208 is configured for displaying, to the user, the remaining usable lifetime of the consumable 202, as determined by the controller 208 at block 303. In some embodiments, the controller 208 may be configured for determining and displaying the remaining usable lifetime of the consumable 202 in response to receiving user input/request.

### Conclusion

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value.

Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful and is not intended to exclude other embodiments from the scope of the disclosure, or from the scope of the claims.

1-251. (canceled)

252. A smoking substitute device for consuming a consumable in a session, comprising:
a display; and
a controller configured to determine a remaining usable lifetime of the consumable in said session, and display said remaining usable lifetime in predetermined intervals at the display.

253. The device according to claim 252, wherein the controller is configured to determine the remaining usable lifetime of the consumable based on remaining time left in the session.

254. A smoking substitute device for consuming a consumable in a session, comprising:
a display; and
a controller configured to determine, based on the type of consumable, the remaining usable lifetime of the consumable in said session, and display said remaining usable lifetime at the display.

255. The device according to claim 254, wherein the controller is configured to determine the total duration of the consumable cycle based on the type of consumable, and further determine the remaining usable lifetime of the consumable in proportion to the total duration of the consumable cycle.

256. The device according to claim 254, wherein the controller is configured to determine and display the remaining usable lifetime of the consumable at the display in predetermined intervals.

257. The device according to claim 252, wherein the controller is further configured to receive a user input and determine the remaining usable lifetime of the consumable in response to receiving the user input.

258. The device according to claim 252, wherein the controller is configured to detect and display the remaining usable lifetime of the consumable on the display during an active smoking session.

259. The device according to claim 252, wherein the controller is configured to determine the remaining usable lifetime of the consumable in proportion to total duration of the smoking session.

260. The device according to claim 252, wherein the controller is configured to measure ambient temperature and incorporate the ambient temperature into the determination of the remaining usable lifetime of the consumable.

261. The device according to claim 252, wherein the controller is configured to generate an alert to the user to indicate that the user is entering a final time period of usable lifetime of the consumable based on the determination of the remaining usable lifetime of the consumable, optionally wherein the alert is provided in the form of audio or haptic feedback.

262. The device according to claim 252, wherein the controller is configured to:
determine a number and/or magnitude of puff inhalations made by the user during an active smoking session; and
incorporate this number into the determination of the remaining usable lifetime of the consumable.

263. A method of consuming a consumable in a session with a smoking substitute device comprising a display and a controller, the method comprising:
determining the remaining usable lifetime of the consumable to be consumed in said session; and
displaying said remaining usable lifetime of the consumable in predetermined intervals on the display.

264. (canceled)

265. A method of consuming a consumable in a session with a smoking substitute device comprising a display and a controller, the method comprising:
determining the remaining usable lifetime of the consumable to be consumed in said session, in response to determining type of consumable; and
displaying said remaining usable lifetime of the consumable on the display.

266. The device according to claim 252, wherein the display comprises a discrete number of display regions corresponding to the predetermined intervals, and wherein the controller is configured to activate a number of the display regions to indicate the remaining usable lifetime, optionally wherein the display comprises four display regions to indicate the remaining usable lifetime in predetermined intervals of 25%.

267. The device according to claim 252, wherein the controller is further configured to determine a remaining battery life of the device and display said battery life on the display when the device is in a standby mode.

268. The device according to claim 266, wherein the controller is further configured to determine a remaining battery life of the device and display said battery life on the display in predetermined intervals using the display regions when the device is in a standby mode.

269. The device according to claim 252, wherein the controller is configured to adjust a length of the smoking session based on a/the type of the consumable.

270. The device according to claim 252, wherein the consumable is a heat-not-burn consumable.

271. The device according to claim 252, comprising a memory coupled with the controller, wherein the memory is pre-programmed to store an expected lifetime of the consumable, wherein the controller is further configured to provide a smoking session of a duration corresponding with the expected lifetime of the consumable and to monitor time elapsed during said session, in which the consumable has been consumed, wherein the controller is further configured to determine the remaining usable lifetime of the consumable in said session, based on said expected lifetime and said monitoring.

272. A smoking substitute system comprising the device according to claim 252 and the consumable.
